(19) United States
(12) Patent Application Publication
(10) Pub. No.: US 2026/0363844 A1
(43) Pub. Date: Nov. 19, 2026

(54) SYSTEM FOR TREATING AN AORTIC ANEURYSM AND/OR DISTRIBUTING BLOOD FLOW DURING A MEDICAL PROCEDURE

(71) Applicant: THE COOPER HEALTH SYSTEM, A NEW JERSEY NON-PROFIT CORPORATION, Camden, NJ (US)

(72) Inventor: Joseph V. LOMBARDI, Turnersville, NJ (US)

(73) Assignee: THE COOPER HEALTH SYSTEM, A NEW JERSEY NON-PROFIT CORPORATION, Camden, NJ (US)

(21) Appl. No.: 19/584,186

(22) PCT Filed: Nov. 1, 2024
(86) PCT No.: PCT/US2024/054191

§ 371 Date: Jun. 15, 2026

Related U.S. Application Data
(60) Provisional application No. 63/595,249, filed on Nov. 1, 2023.

(51) Int. Cl. A61F 2/07; A61M 1/36; A61M 39/22

(52) U.S. Cl. CPC A61F 2/07; A61M 1/36; A61M 39/22; A61F 2002/077; A61F 2230/0069

(57) ABSTRACT

Systems, methods, medical devices, and kits. The disclosure includes systems, methods, medical devices, and kits for repairing an aortic aneurysm comprising a perfusion apparatus and an introducer sheath apparatus. The perfusion apparatus comprising an inlet conduit having a proximal end configured to be fluidly coupled to the distal end of the perfusion branch conduit, a plurality of secondary conduits branching from the inlet conduit and configured for fluid coupling to aortic branches, and a valve assembly configured to selectably alter the plurality of secondary conduits between an open flow state and a closed sealed state.

### CROSS REFERENCE TO RELATED APPLICATIONS

This application claims the benefit of U.S. Provisional Application 63/595,249, which was filed Nov. 1, 2023, is titled THORACOAORTIC HYBRID GRAFT AND SURGICAL METHODS THEREOF, and is incorporated herein by reference as if fully set forth.

### FIELD

The disclosure relates to systems, methods, medical devices and kits. Uses for the systems, methods, medical devices and kits include those pertaining to repairing an aorta.

### BACKGROUND

Surgical repairs involving stents have become the standard when treating aortic aneurysms and recent advances have led to the use of hybrid stent-grafts. While use of such technology has proven revolutionary in treating aneurysms in the aortic arch, it has been harder to utilize the technology in repairing the more invasive thoracoabdominal aortic aneurysms. In addition, surgeons are still often required to utilize heart bypass techniques which increase the difficulty of successfully performing these already arduous surgical repairs. Thus, there is a need in the art for a hybrid stent-graft with an attached perfusion apparatus that allows a surgeon to repair a thoracoabdominal aneurysm while minimizing the need for a heart bypass.

### BRIEF SUMMARY

In some embodiments, the disclosure relates to a system for repairing an aortic aneurysm in an aorta of a patient. In some embodiments, the system comprises a perfusion apparatus comprising an inlet conduit comprising a proximal end, and a plurality of secondary conduits branching from the inlet conduit and configured for fluid coupling to aortic branches. In some embodiments, the system further comprises a valve assembly configured to selectably alter the plurality of secondary conduits between an open flow state and a closed sealed state. In some embodiments, the system further comprises an introducer sheath apparatus configured to be fluidly coupled to the inlet conduit of the perfusion apparatus to deliver blood from the aorta or an inflow artery to the inlet conduit.

In some embodiments, the disclosure relates to a method of repairing an aortic aneurysm in a subject comprising a heart, an aorta, another inflow artery, and a site of the aortic aneurysm. In some embodiments, the method comprises inserting an introducer sheath into at a site between the heart and the site of the aortic aneurysm or into the other inflow artery. In some embodiments, the method further comprises fluidly coupling the introducer sheath to an inlet conduit of a perfusion apparatus. In some embodiments, the inlet conduit comprises a proximal end and the perfusion apparatus further comprising a plurality of secondary conduits branching from the inlet conduit. In some embodiments, each secondary conduit comprises a respective tip distal to the inlet conduit. In some embodiments, one or more or all of the respective tips is configured for fluid coupling to aortic branches. In some embodiments, the perfusion apparatus further comprises a valve assembly configured to selectably alter the plurality of secondary conduits between an open flow state and a closed sealed state. In some embodiments, the method further comprises inserting a plurality of second introducer sheath apparatuses into different aortic branches of the subject. In some embodiments, the method further comprises connecting each of the plurality of secondary conduits to a respective one of the plurality of second introducer sheath apparatuses.

In some embodiments, the disclosure relates to a system for repairing an aortic aneurysm. In some embodiments, the system comprises a vascular prosthesis. In some embodiments, the vascular prosthesis comprises a primary conduit forming a primary lumen extending from a primary inlet opening to a primary outlet opening. In some embodiments, the primary conduit is configured to be fluidly coupled to an aorta of a patient. In some embodiments, the system further comprises a perfusion apparatus. In some embodiments, the perfusion apparatus comprises an inlet conduit having a proximal end; a plurality of secondary conduits branching from the inlet conduit and configured for fluid coupling to aortic branches. In some embodiments, the system further comprises a valve assembly configured to selectably alter the plurality of secondary conduits between an open flow state and a closed sealed state. In some embodiments, the system further comprises an introducer sheath apparatus configured to be fluidly coupled to the inlet conduit of the perfusion apparatus so that blood can flow through the introducer sheath apparatus and into the inlet conduit of the perfusion apparatus.

In some embodiments, the disclosure relates to a medical device for distributing blood from an aorta or another inflow artery of a patient to a plurality aortic branches. In some embodiments, the medical device comprises an introducer sheath sub-system. In some embodiments, the introducer sheath sub-system comprises a sheath tube forming a working lumen for receiving blood directly from the aorta or other inflow artery and a port in fluid cooperation with the working lumen. In some embodiments, the medical device further comprises a perfusion sub-system. In some embodiments, the perfusion sub-system comprises an inlet conduit having a proximal end fluidly coupled to the port of the introducer sheath subs-system to receive blood flowing through the sheath tube from the aorta or other inflow artery. In some embodiments, the perfusion sub-system further comprises a plurality of secondary conduits branching from the inlet conduit. In some embodiments, each of the plurality of secondary conduits comprises a distal end configured for fluid coupling to one of the aortic branches to deliver blood flowing through the inlet conduit to the one of the aortic branches. In some embodiments, the medical device further comprises a valve assembly configured to selectably alter the plurality of secondary conduits between an open flow state and a closed sealed state.

In some embodiments, the disclosure relates to a method of repairing an aortic aneurysm in a damaged section of an aorta. In some embodiments, the method comprises a) preventing the flow of blood in the aorta from reaching the damaged section of the aorta. In some embodiments, the method further comprises b) routing blood from a first section of the that is upstream of the damaged section or from another inflow artery to a plurality of aortic branches via a medical device. In some embodiments, the medical device comprises an introducer sheath sub-system and a perfusion sub-system. In some embodiments, the introducer sheath sub-system receives blood from the first section of the aorta or other inflow artery via a sheath tube that penetrates an aortic wall of the first section of the aorta or a wall of the other inflow artery and the perfusions subsystem distributes the blood flowing from the sheath tube to a plurality of secondary conduits having distal ends that are fluidly coupled to the plurality of aortic branches. In some embodiments, the method further comprises c) implanting a vascular prosthetisis in the damaged section.

In some embodiments, the disclosure relates to a medical device for distributing blood from an aorta or other inflow artery of a patient to a plurality aortic branches. In some embodiments, the medical device comprises an introducer sheath sub-system comprising a plurality of introducer sheath apparatuses. In some embodiments, each of the plurality of introducer sheath apparatuses comprises a sheath tube forming a working lumen for flowing blood directly into one of the aortic branches and a port in fluid cooperation with the working lumen. In some embodiments, the medical device further comprises a perfusion sub-system comprising an inlet conduit having a proximal end for receiving blood from the aorta or other inflow artery, and a plurality of secondary conduits branching from the inlet conduit. In some embodiments, each of the plurality of secondary conduits comprises a distal end fluidly coupled to one of the plurality of the introducer sheath apparatuses of the introducer sheath sub-system. In some embodiments, the medical device further comprises a valve assembly configured to selectably alter the plurality of secondary conduits between an open flow state and a closed sealed state.

In some embodiments, the disclosure relates to a system for repairing an aortic aneurysm. In some embodiments, the system comprises a vascular prosthesis. In some embodiments, the vascular prosthesis comprises a primary conduit forming a primary lumen extending from a primary inlet opening to a primary outlet opening. In some embodiments, the primary conduit is configured to be fluidly coupled to an aorta of a patient. In some embodiments, the vascular prosthesis further comprises a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen. In some embodiments, the system further comprises a perfusion apparatus comprising an inlet conduit having a proximal end configured to be fluidly coupled to the distal end of the perfusion branch conduit. In some embodiments, the perfusion apparatus further comprises a plurality of secondary conduits branching from the inlet conduit and having a distal end. In some embodiments, the medical device further comprises a valve assembly configured to selectably alter the plurality of secondary conduits between an open flow state and a closed sealed state. In some embodiments, the medical device further comprises a plurality of introducer sheath apparatuses. In some embodiments, each of the plurality of introducer sheath apparatuses is configured to be fluidly coupled to the distal end of one of the plurality of secondary conduits to deliver blood from the secondary conduit to a respective one of the aortic branches.

An aspect may be directed to a surgical kit for repairing an aortic aneurysm and components thereof. In an embodiment, the surgical kit comprises a vascular prosthesis. The vascular prosthesis comprises a primary lumen extending from a primary inlet opening to a primary outlet opening. The primary lumen is configured to be fluidly coupled to the aorta of a patient. The vascular prosthesis further comprises a perfusion branch conduit forming a perfusion branch lumen extending from a perfusion branch opening in the primary lumen to a perfusion outlet opening at a distal end of the perfusion branch conduit. The perfusion branch opening is in fluid communication with the primary lumen. In some embodiments, the vascular prosthesis further comprises a plurality of branch conduits, each of the branch conduits forming a branch lumen extending from an opening in the primary lumen to a branch outlet opening, the branch inlet opening in fluid communication with the primary lumen, and a distal end of each of the branch conduits configured to be fluidly coupled to an aortic branch.

In some embodiments, the surgical kit further comprises a perfusion apparatus comprising an inlet conduit having a proximal end configured to be fluidly coupled to the distal end of the perfusion branch conduit; a plurality of secondary conduits branching from the inlet conduit and configured for fluid coupling to aortic branches; and a valve assembly configured to selectably alter the plurality of secondary conduits between an open flow state and a closed sealed state.

In some embodiments, the primary conduit of the vascular prosthesis comprises a stent-graft portion and a tube-graft portion that meet at a junction region, the stent-graft portion comprising the primary inlet opening and the tube graft portion comprising the primary outlet opening. In certain embodiments, the stent-graft portion comprises a flexible biocompatible membrane attached to a support structure, the stent-graft portion configured to be capable of being radially expanded from a collapsed state to a deployed state.

In some embodiments, the surgical kit further comprises a package containing vascular prosthesis, the perfusion apparatus, and the valve assembly.

In some embodiments, the distal end of the perfusion branch conduit comprises a first quick connect feature and the proximal end of the inlet conduit of the perfusion apparatus comprises a second quick connect feature configured to be matable with the first quick connect feature to fluidly couple the perfusion branch lumen and a passageway of the inlet conduit. In some embodiments, the first and second quick connect features are selected from a group consisting of bayonet connector component, a threaded connector component, a snap-fit connector component, an interference fit connector component, and combinations thereof.

In some embodiments, the disclosure relates to a vascular prosthesis comprising: a primary conduit forming a primary lumen extending from a primary inlet opening to a primary outlet opening, the primary conduit configured so that the primary inlet opening can be fluidly coupled to a portion of an aorta upstream of an aneurysm and the primary outlet opening can be fluidly coupled to a portion of an aorta downstream of an aneurysm; a plurality of branch conduits, each of the branch conduits forming a branch lumen extending from a branch inlet opening in the primary lumen to a branch outlet opening, the branch inlet opening in fluid communication with the primary lumen, and a distal end of each of the branch conduits configured to be fluidly coupled to an aortic branch; and a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen, and the distal end of the perfusion branch conduit comprising a first quick connect feature.

In some embodiments, the first quick connect feature is selected from a group consisting of bayonet connector component, a threaded connector component, a snap-fit connector component, an interference fit connector component, and combinations thereof. In another embodiment, the perfusion branch opening is located closer to the primary inlet opening than any of the branch conduits.

In some embodiments, the disclosure relates to a vascular prosthesis comprising: a primary conduit comprising a primary lumen, extending from a primary inlet opening to a primary outlet opening; a plurality of branch inlet openings; a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen.

In some embodiments, the disclosure relates to a vascular prosthesis comprising: a primary conduit comprising a primary lumen extending from a primary inlet opening to a primary outlet opening, the primary conduit configured so that the primary inlet opening can be fluidly coupled to a portion of an aorta upstream of an aneurysm and the primary outlet opening can be fluidly coupled to a portion of an aorta downstream of an aneurysm; four branch conduits, each of the four branch conduits forming a branch lumen extending from a branch inlet opening in the primary conduit to a branch outlet opening, the branch inlet opening in fluid communication with the primary lumen, and a distal end of each of the four branch conduits configured to be fluidly coupled to an aortic branch; a perfusion branch conduit forming a perfusion branch lumen extending from a perfusion branch opening in the primary lumen to a perfusion outlet opening at a distal end of the perfusion branch conduit, the perfusion branch opening in fluid communication with the primary lumen.

In some embodiments, the disclosure relates to a method of repairing an aortic aneurysm, the method comprising: fluidly coupling: (i) a proximal end of a primary lumen of a vascular prosthesis to a first portion of an aorta of a patient upstream of the aortic aneurysm; and (ii) a distal end of the primary lumen of the vascular prosthesis to a second portion of the aorta downstream of the aortic aneurysm, wherein one or more aortic branches of the patient are located between the first and second portions of the aorta, thereby allowing blood to flow through the primary lumen; fluidly coupling an inlet conduit of a perfusion apparatus to a distal end of a perfusion branch conduit of the vascular prosthesis, a valve assembly of the perfusion apparatus being in a closed sealed state such that blood from the primary lumen of the vascular prosthesis cannot flow through a plurality of secondary conduits of the perfusion apparatus that branch from the inlet conduit; fluidly coupling distal ends of the secondary conduits to the one or more aortic branches; and altering the valve assembly to an open flow state to restore blood flow to the one or more aortic branches.

### BRIEF DESCRIPTION OF THE DRAWINGS

The detailed description will be better understood when read in conjunction with the appended drawings. It should be understood, however, that the invention is not limited to the precise arrangements and instrumentalities of the embodiments shown in the drawings.

FIG. 1 illustrates a surgical kit;

FIG. 2 illustrates a vascular prosthesis;

FIG. 3 illustrates a cross sectional view of the vascular prosthesis of FIG. 2;

FIG. 4 illustrates a perfusion apparatus;

FIG. 5 illustrates the perfusion apparatus shown in FIG. 4 attached to the vascular prosthesis shown in FIGS. 2 and 3;

FIGS. 6A and 6B illustrate a coupling mechanism between the vascular prosthesis and perfusion apparatus as shown in FIG. 5;

FIG. 7 illustrates the portion of the aorta where a vascular prosthesis may be implanted;

FIGS. 8A through 8M illustrate a surgical method;

FIGS. 9A and 9B illustrate a vascular prosthesis;

FIG. 10 illustrates a surgical kit;

FIG. 11 illustrates a surgical kit;

FIG. 12 illustrates a surgical kit;

FIG. 13 illustrates a perfusion apparatus;

FIG. 14A illustrates a perfusion apparatus and an introducer sheath apparatus;

FIG. 14B illustrates a perfusion apparatus and an introducer sheath apparatus;

FIGS. 15A and 15BB illustrate an exemplary introducer sheath apparatus;

FIG. 16 illustrates a surgical kit;

FIG. 17 illustrates an introducer sheath apparatus;

FIG. 18 illustrates an introducer sheath apparatus;

FIG. 19 illustrates an introducer sheath apparatus and a perfusion apparatus inlet conduit;

FIG. 20 illustrates an introducer sheath apparatus and a perfusion apparatus inlet conduit; and

FIG. 21 illustrates a primary connection feature and a port connection feature.

### DETAILED DESCRIPTION

The following description of the embodiment(s) is exemplary in nature. The description of some embodiments is intended to be read in connection with the accompanying drawings, which are to be considered part of the entire written description. In the description of embodiments herein, any reference to direction or orientation is merely intended for convenience of description and is not intended in any way to limit the scope of the embodiment illustrated. Relative terms such as “lower,” “upper,” “horizontal,” “vertical,” “above,” “below,” “up,” “down,” “left,” “right,” “top,” “bottom,” “front” and “rear” as well as derivatives thereof (e.g., “horizontally,” “downwardly,” “upwardly,” etc.) should be construed to refer to the orientation as then described or as shown in the drawing under discussion. These relative terms are for convenience of description only and do not require a particular orientation unless explicitly indicated as such. Terms such as “attached,” “affixed,” “connected,” “coupled,” “interconnected,” “secured” and other similar terms refer to a relationship wherein structures are secured or attached to one another either directly or indirectly through intervening structures, as well as both movable or rigid attachments or relationships, unless expressly described otherwise. The discussion herein describes and illustrates some possible non-limiting combinations of features that may exist alone or in other combinations of features. Furthermore, as used herein, the term “or” is to be interpreted as a logical operator that results in true whenever one or more of its operands are true.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise define, all technical and scientific terms used herein have the same meaning as commonly understood to one of ordinary skill in the art. As used herein, the term “proximal” and “distal” refer to a location or portion of a device that, when inserted, is upstream or downstream with respect to blood flow.

The below description illustrates embodiments herein with reference to repairing an aorta, aortic aneurysm or the like. However, embodiments herein may be used in the context of managing or redirecting blood flow in a variety of surgical or medical situations. Thus, Embodiments herein also include systems, methods, medical devices, and kits related to managing or redirecting blood flow.

In order to better understand the embodiments herein reference is made to the accompanying drawings in which the corresponding parts are indicated by the same reference numbers throughout the drawings.

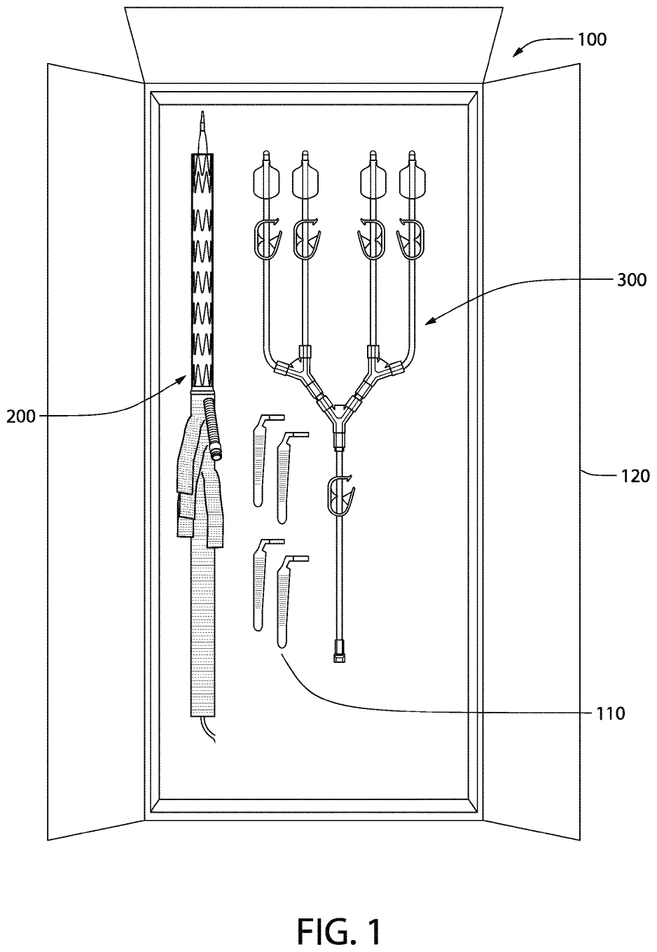

Referring to FIG. 1, a surgical kit 100 is disclosed according to an embodiment. The surgical kit 100 generally comprises a vascular prosthesis 200, a perfusion apparatus 300, and at least one surgical accessory 110, all of which are provided in a package 120. The package may be a sealed container that maintains the sterile state of the contents, such as sealed plastic bag, box, or the like.

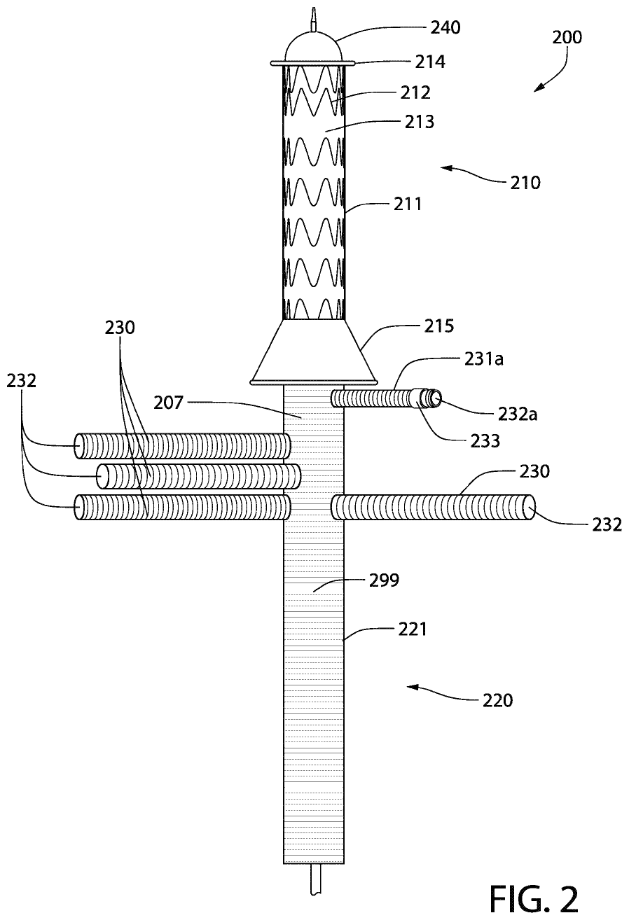

In some embodiments, the surgical kit 100 may further comprise a molding balloon 240 (FIG. 2). The surgical accessories 110 may be tools for cutting, dissecting, suturing, hemostasis, holding, retracting, disinfecting, or soaking. In some embodiments, the surgical accessories 110 may be scalpels, forceps, needles, needle holders, threads, retractors, suction tips, staplers, clips, scissors, hooks, dissectors, hemostats, gauze, saline, trocars, containers, trays, or any combination thereof.

The at least one surgical accessory 110 may be present in any quantity. In some embodiments, the surgical accessories 110 includes at least one clamp 110. While FIG. 1 shows a clamp 110 as the surgical accessory, a person of skill in the art would understand that the surgical kit 100 may include other types of surgical accessories. As mentioned above, the surgical kit 100 comprises the package 120 containing the vascular prosthesis 200, the perfusion apparatus 300, and other component, such as the clamps 110. In some embodiments, the package 120 may further comprise the molding balloon 240.

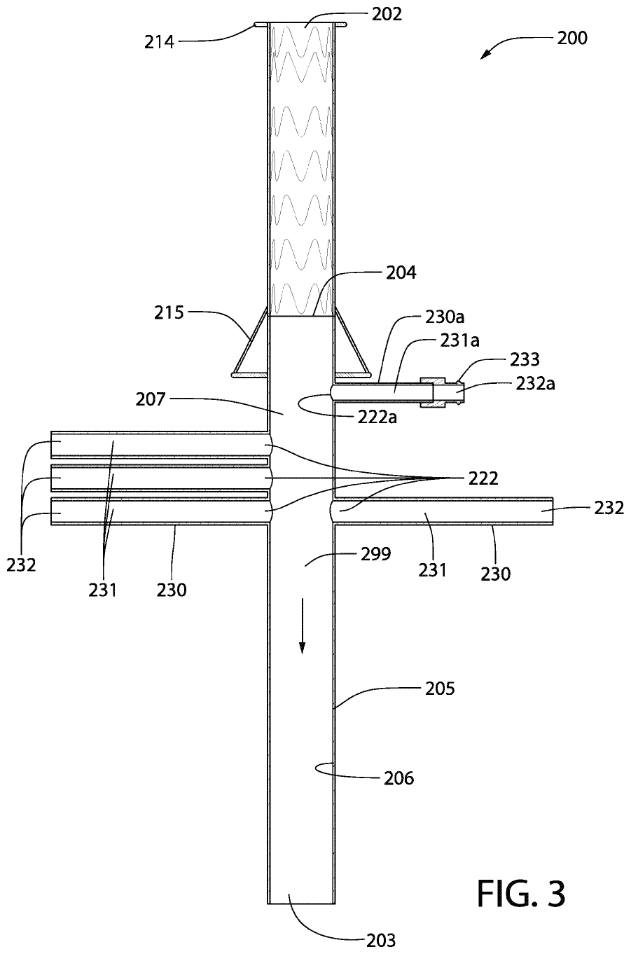

Referring now to FIGS. 2 and 3, the vascular prosthesis 200 will be further described. The vascular prosthesis 200 generally comprises a proximal stent-graft portion 210 and a distal tube-graft portion 220. The vascular prosthesis 200 comprises a primary conduit 299. The primary conduit 299 is a tubular structure having a generally cylindrical transverse cross-sectional profile. The primary conduit 299 has an outer surface 205 and an inner surface 206. The inner surface 206 of the prosthesis 200 defines a primary lumen 201 that extends longitudinally through the vascular prosthesis 200 from a primary inlet opening 202 at the proximal end of the primary conduit 299 to a primary outlet opening 203 at the distal end of the primary conduit 299. The primary conduit 299 is configured to be fluidly coupled to an aorta of a patient to allow fluid such as blood to pass through the primary lumen 201 in a manner similar to the natural lumen of the aorta. As can be seen, a proximal section of the primary conduit 299 is part of the stent-graft portion 210 of the vascular prosthesis 200 while a distal section of the primary conduit 299 is part of the tube graft portion 220. The stent-graft portion 210 of the primary conduit 299 and tube graft portion 220 of the primary conduit 299 are joined together at a junction region 204, which is located between the proximal and distal ends of the primary conduit 299.

The stent-graft portion 210 of the primary conduit 299 is a compressible tube 211 comprising a support structure 212 and a flexible membrane 213. The support structure 212 is configured to provide radial support to the stent-graft portion 210 and to radial expand to conform to the diameter of the surrounding blood vessel when deployed. The support structure 212 may be made of any biocompatible metal, plastic, or derivatives thereof. In some embodiments, the support structure 212 may be made from stainless steel, cobalt chromium, or nitinol. In some embodiments, the support structure 212 may comprise a plurality of support elements. The plurality of support elements may be unconnected, partially interconnected, or fully interconnected. In other embodiments, the support structure 212 may comprise a single support element. The support structure 212 may be a spring, wire, mesh, or any other configuration known in the art. In one embodiment, the support structure 212 is a metal mesh. In some embodiments, the support structure 212 is configured to provide radial support to the stent-graft portion 210.

The membrane 213 is attached to the support structure 212 and configured to be able to radially expand in conjunction with the support structure 212. In some embodiments, the membrane 213 is attached using a thermal process or sewing. The membrane 213 may be made from any flexible biocompatible material that forms a seal. In some embodiments, the membrane 213 is made from woven polyester or derivative thereof. In some embodiments, the membrane 213 may be made from polyester or polyester derivative, polyaramids, polyacrylonitrile, nylon, cellulose, polyurethane, polyethylene terephthalate (PET/Dacron), polytetrafluoroethylene (PTFE), or expanded polytetrafluoroethylene (ePTFE), and thermoplastic polyethylenes such as ultra high molecular weight polyethylene (UHMWPE) or high-modulus polyethylene (HMPE). The membrane 213 may further comprise angiographic markers. The markers may be made from any radiopaque material known in the art. In some embodiments, the angiographic markers may be made from platinum, platinum-iridium, tantalum, gold, or any combination thereof. In some embodiments, the stent-graft portion 210 is comprised solely of a support structure 212 without a membrane 213.

In the exemplified embodiment, the stent-graft portion 210 of the primary conduit 299 comprises a hemostatic collar 214. The hemostatic collar 214 is a flange or ring-like structure that protrudes radially from the proximal end of the primary conduit 299. The hemostatic collar 214 is exemplified as being located at the proximal end of the primary conduit 299 but in other embodiments may be spaced therefrom. The hemostatic collar 214 can be made from any biocompatible material known in the art. In some embodiments, the hemostatic collar 214 is made from biocompatible plastic, metal, or any derivative thereof. In some embodiments, the hemostatic collar 214 is made from biocompatible felt such as polyglycolic acid felt (PGA) or PTFE felt. The hemostatic collar 214 is configured to secure the proximal end of the prosthesis 200 within the aorta. In some embodiments, the hemostatic collar 214 may further comprise interspersed projections or barbs which are configured to prevent the vascular prosthesis 200 from migrating.

In some embodiments, the stent-graft portion 210 may also comprise a hemostatic apron 215. The hemostatic apron 215 is located near the junction region 204. In the exemplified embodiment, the hemostatic apron 215 traverses the junction region 204 such that an upper portion of the hemostatic apron 215 is located above the junction region 204 and a lower portion of the hemostatic apron 215 is located below the junction region 204. The hemostatic apron 215 is connected to the primary conduit 299 above the junction region 204, and extends downwards and outwards to form a generally truncated cone-shaped flange. The hemostatic apron 215 may be made from any biocompatible material known in the art. The apron 215 is configured to be attached to the wall of an aorta in a patient to create an anastomosis between the prosthesis 200 and the aorta to prevent intercostal back bleeding.

The tube graft portion 220 of the primary conduit 299 is a generally cylindrical tube 221 which may comprise a plurality of inlet openings 222 (the purpose of which is discussed in greater detail below). The tube graft portion 220 may be made from any biocompatible material known in the art. In some embodiments, the tube graft portion 220 may be made from polyester or polyester derivative, polyurethane, polyethylene terephthalate (PET/Dacron), polytetrafluoroethylene (PTFE), or expanded polytetrafluoroethylene (ePTFE), thermoplastic polyethylenes such as ultra high molecular weight polyethylene (UHMWPE) or high-modulus polyethylene (HMPE). In some embodiments, the tube graft portion 220 is made from polyethylene terephthalate (PET/Dacron).

The plurality of openings 222 fluidly connect the primary lumen 201 to a plurality of branch lumens 231, which are formed by the branch conduits 230. The plurality of openings 222 may be referred to as branch inlet openings 222 when being discussed in relation to the branch conduits 230. In the exemplified embodiment, the vascular prosthesis 200 further comprises a plurality of branch conduits 230. Each of the branch conduits 230 forms a branch lumen 231 that extends from one of the openings 222 at the proximal end thereof to a branch outlet opening 232 at the distal end thereof. The branch lumens 231 are part of the tube graft portion 220 of the vascular prosthesis 200.

While four branch conduits 230 are shown in the exemplified embodiment, more or less branch conduits 230 can be provided as desired and depending on the surgical requirements. Moreover, in certain embodiment, no branch conduits 230 may be include and only a perfusion branch conduit 230a may be included. When intended to be used to couple to aortic branches of the patient, the branch conduits 230 may be referred to as aortic branch conduits 230 of the vascular prosthesis 200. In certain embodiments, one or more of the branch conduits 230 may be removable or temporary.

The distal end of branch conduits 230 are configured to be fluidly coupled to an aortic branch of a patient. In some embodiments, the branch conduits 230 are configured to be fluidly coupled to visceral arteries. In some embodiments, multiple branch conduits 230 are connected to multiple aortic branches through a single connection. In other embodiments, the branch conduits 230 are connected to aortic branches through separate connections.

The vascular prosthesis 200 further comprises a perfusion branch conduit 230a. To accommodate the perfusion branch conduit 230a the primary conduit 299 comprises a perfusion branch opening 222a in a sidewall thereof. The perfusion branch opening 222a is closer to the proximal end of the primary conduit 299 than any of the openings 222 of the branch conduits 230. The axial section of the primary conduit 299 that is between the perfusion branch opening 222a and the closest of the openings 222 provides a clamping zone 207 so that the primary lumen 201 can be sealed by the application of a clamp to the primary conduit 299 in this axial section (discussed in greater detail below). As exemplified, the perfusion branch conduit 230a is located at the proximal end of the tube-graft portion 220, generally below the junction region 204. The clamping zone 207 is located directly below the perfusion branch conduit 230a and above any other branch conduits 224. The clamping zone 207 is an axial section of the tube-graft portion 220 of the primary conduit 299 that lacks any openings 222.

The perfusion branch conduit 230a forms a perfusion branch lumen 231a which extends from the perfusion branch opening 222a at the proximal end of the perfusion branch conduit 230a to a perfusion outlet opening 232a at the distal end of the perfusion branch conduit 230a. The perfusion branch opening 222a fluidly connects the primary lumen 201 to the perfusion branch lumen 231a. The perfusion outlet opening 232a allows fluid communication between the vascular prosthesis 200 and the perfusion apparatus 300 via a first quick connect feature 233, as discussed in greater detail below.

In certain embodiments, the diameter for the perfusion branch conduit 230a may be greater than the diameter of the branch conduits 230. In one specific example, the diameter for the perfusion branch conduit 230a may be about 10 mm while the diameter of the branch conduits 230 may be in the range of 6 mm to 8 mm.

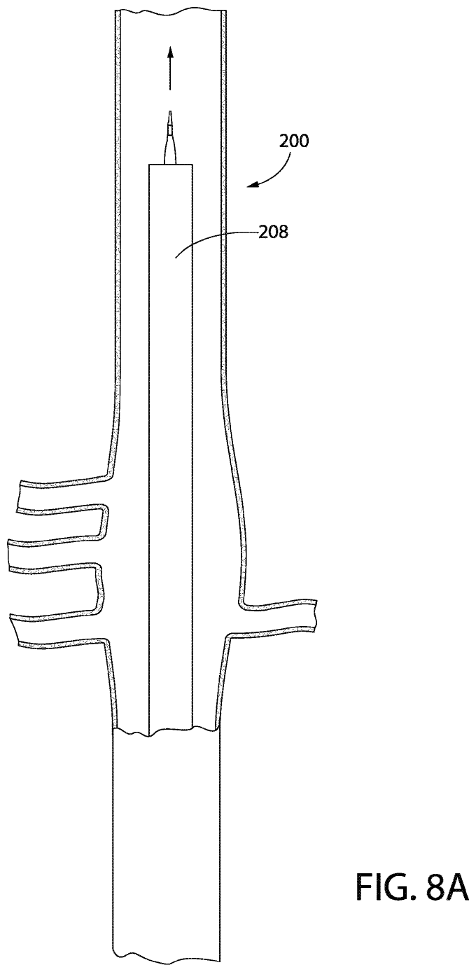

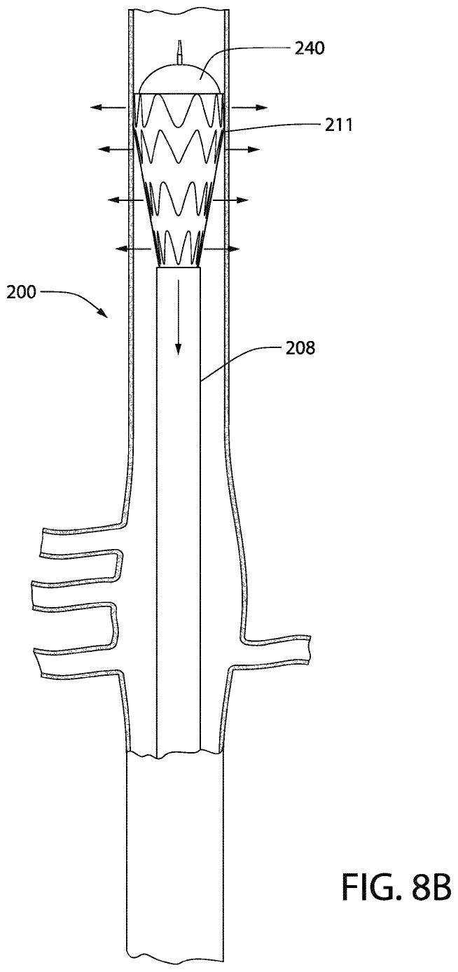

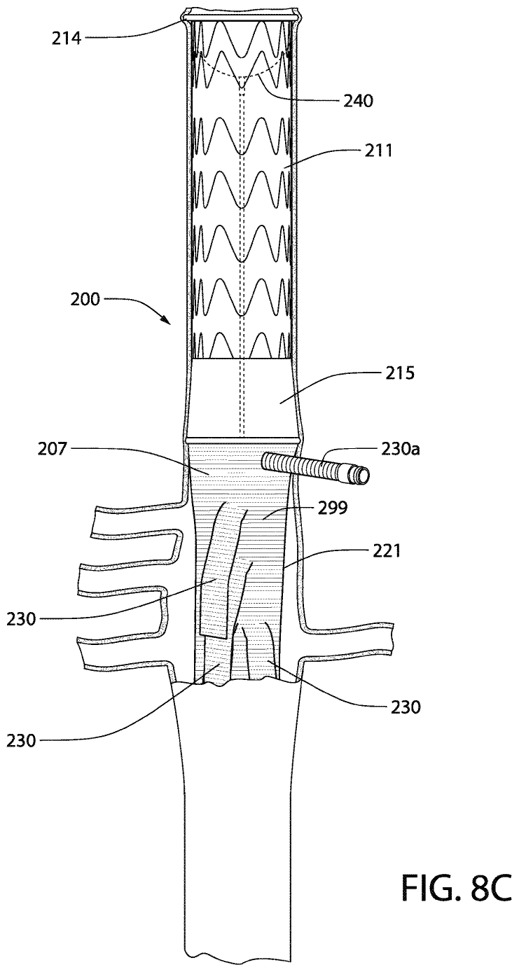

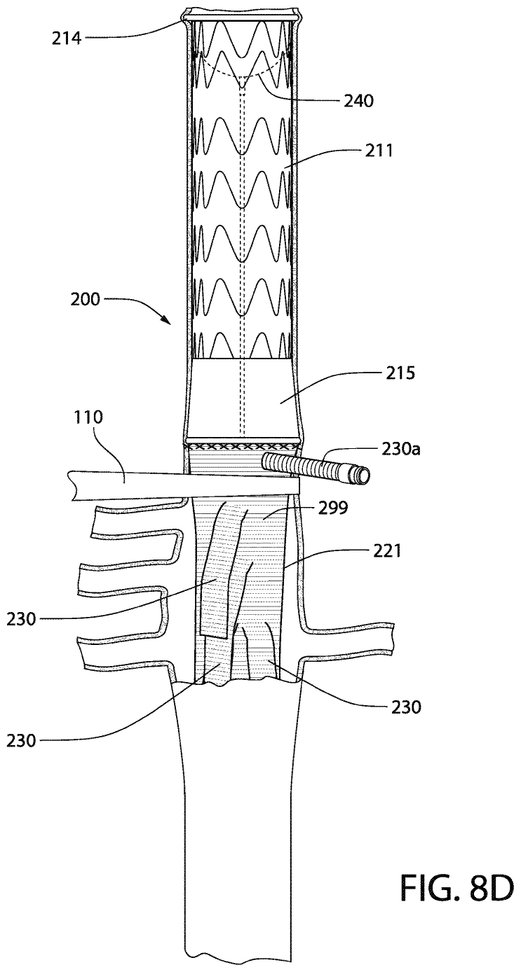

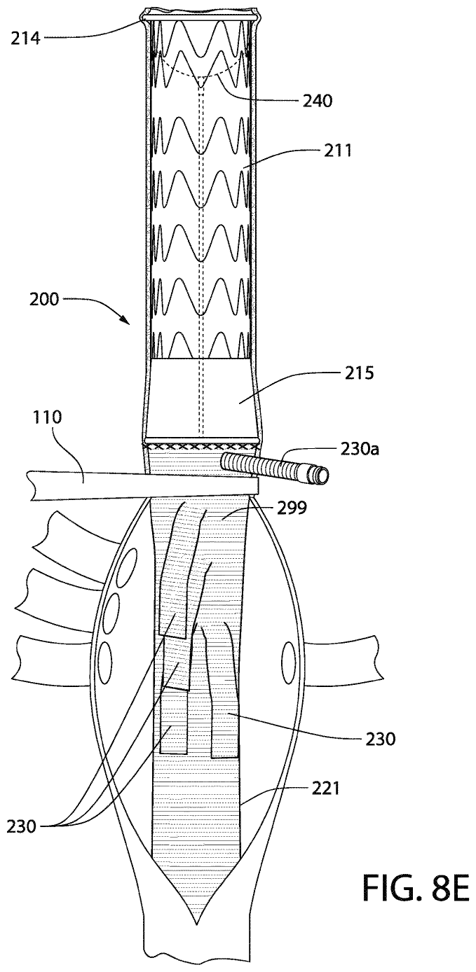

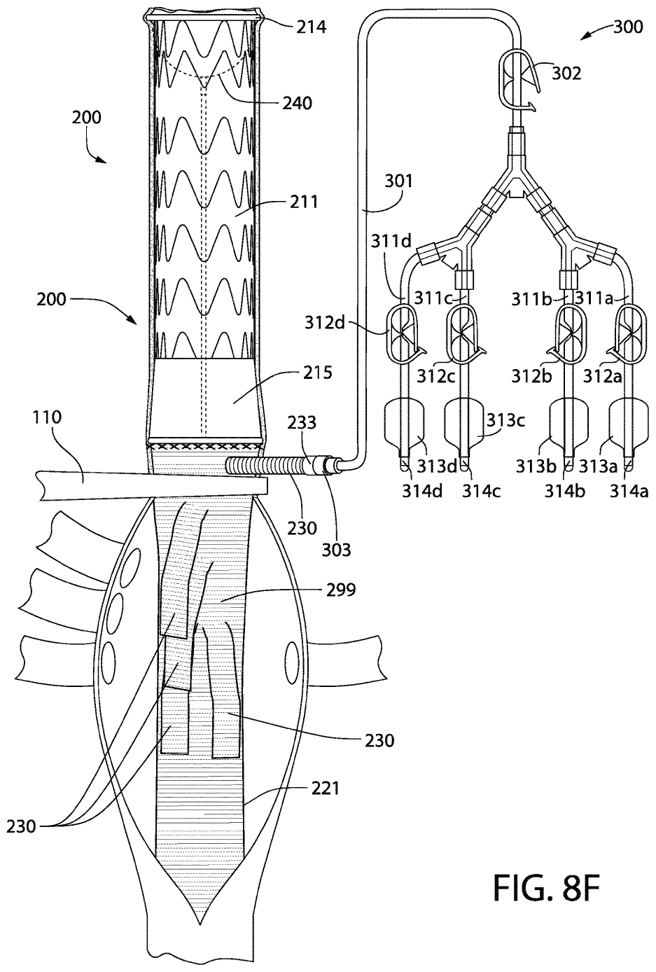

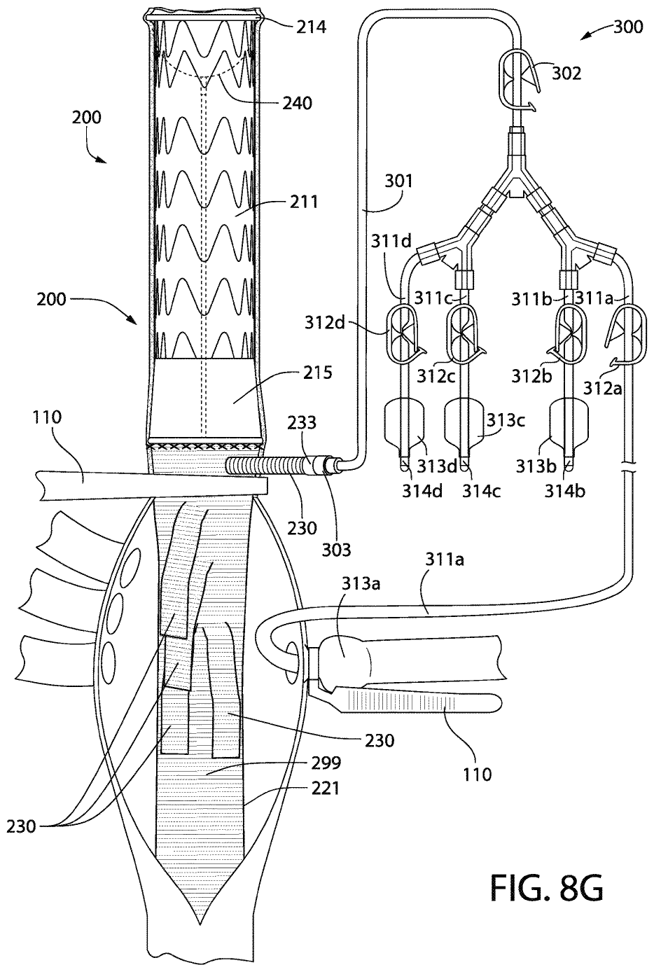

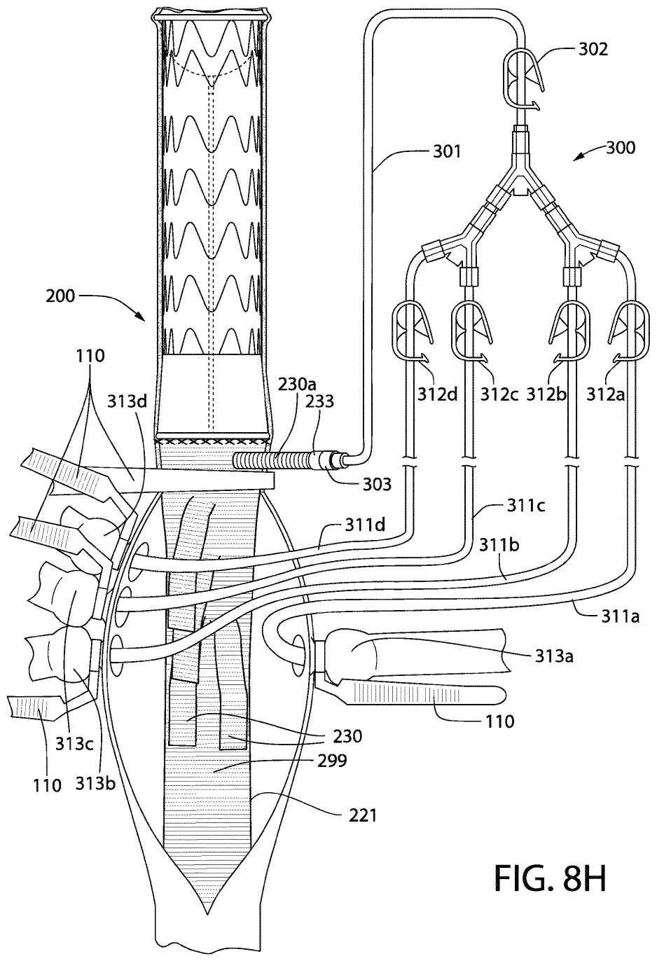

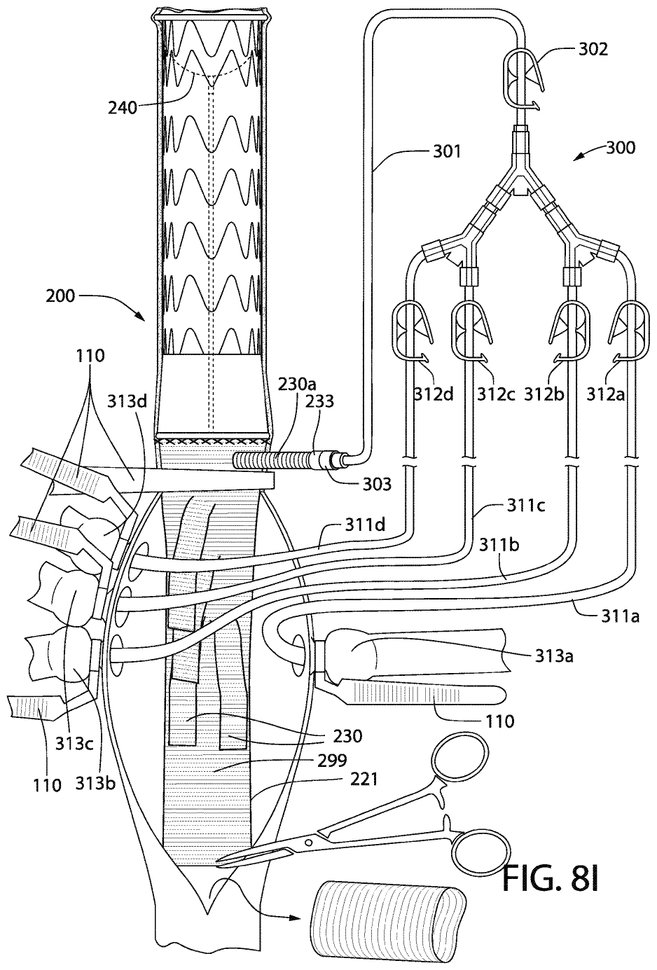

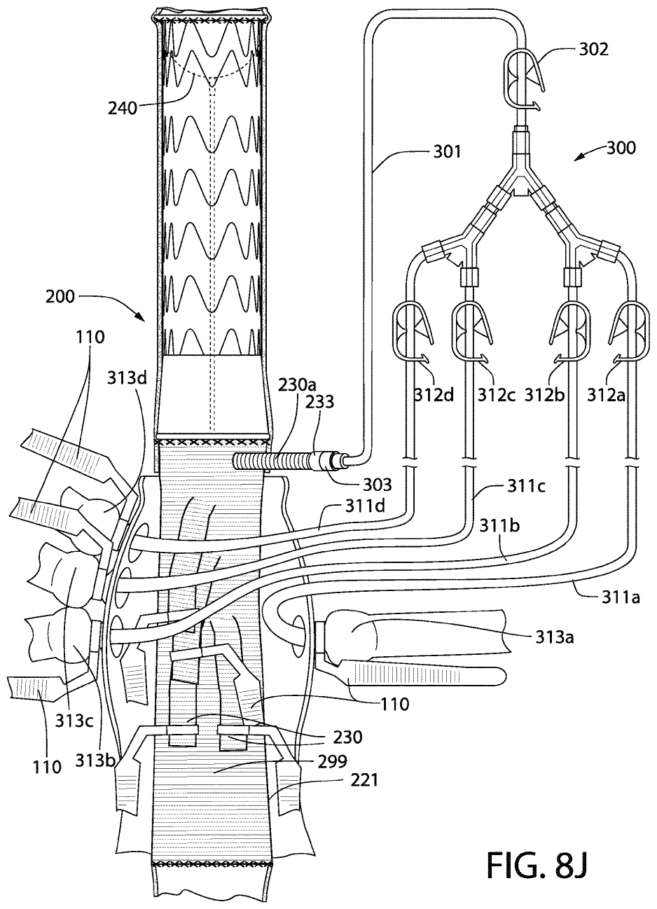

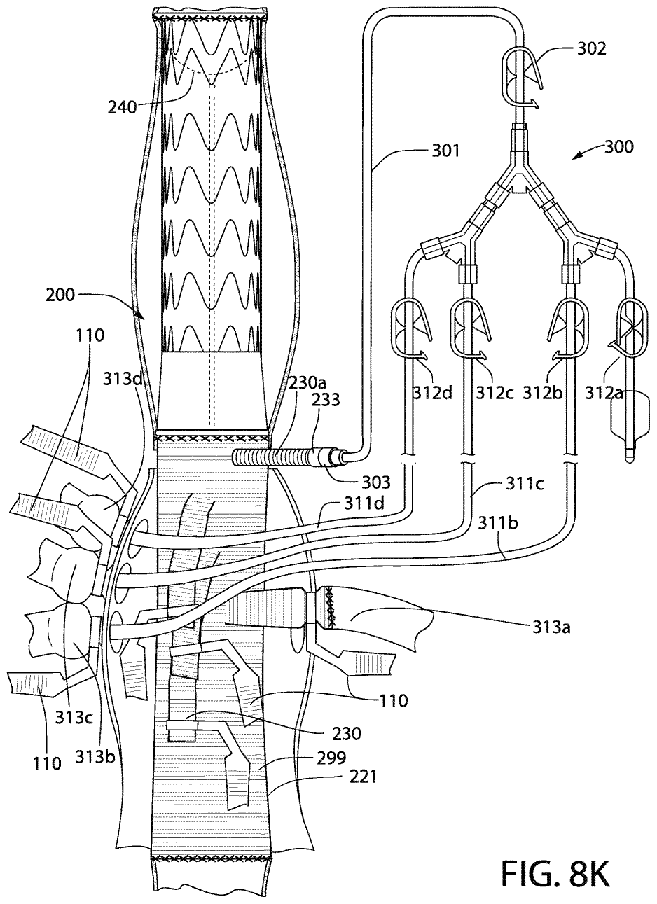

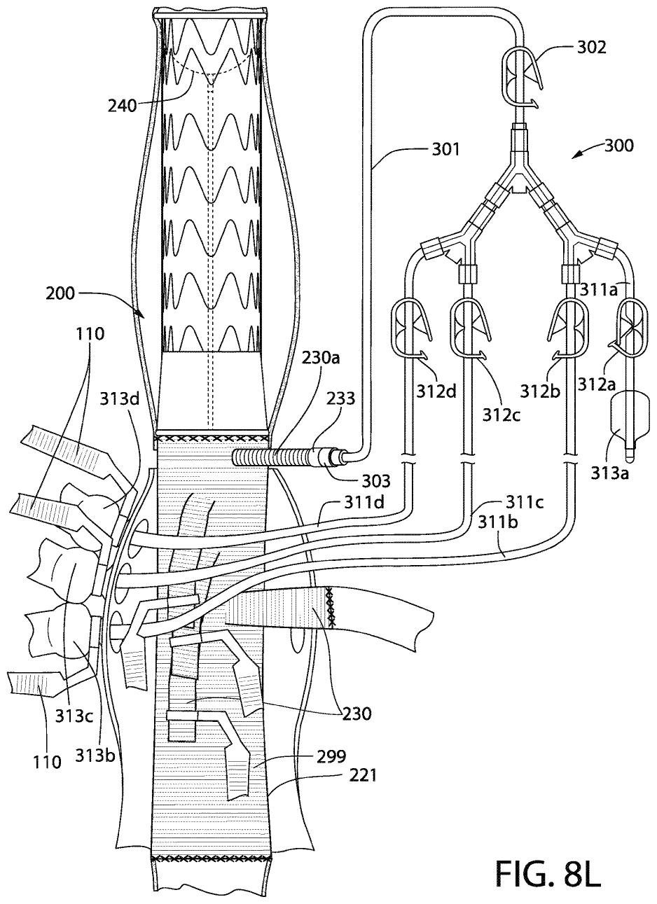

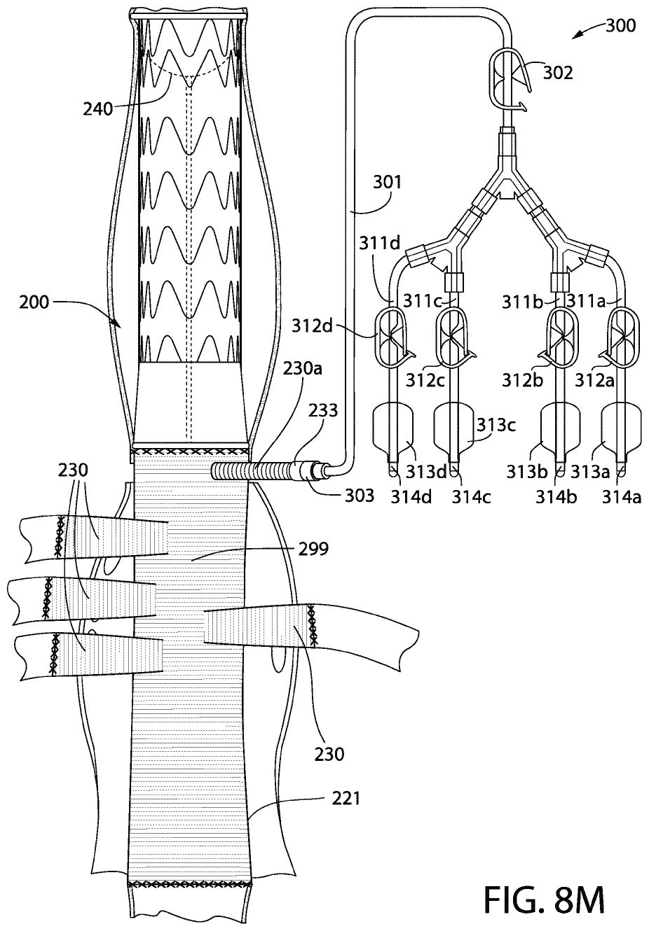

In some embodiments, a molding balloon 240 (see FIG. 8B) is included that is positioned within the primary lumen 201 of the prosthesis 200. The molding balloon 240 is configured to position the vascular prosthesis 200 within the aorta. In certain embodiments, the molding balloon 240 is configured so that, when inflated, the primary lumen 201 is sealed and blood is prevented from flowing from the aorta of the patient and into (or through) the primary lumen 201. When inflated, the molding balloon 240 prevents blood from flowing through the primary lumen 201 such that it would reach the branch conduits 230 or the perfusion branch conduit 230a. Although not shown, one skilled in the art would understand that the vascular prosthesis 200 may comprise additional features not shown in the drawings. For example, the vascular prosthesis 200 may further comprise a guide wire or other deployment devices which are standard in the art.

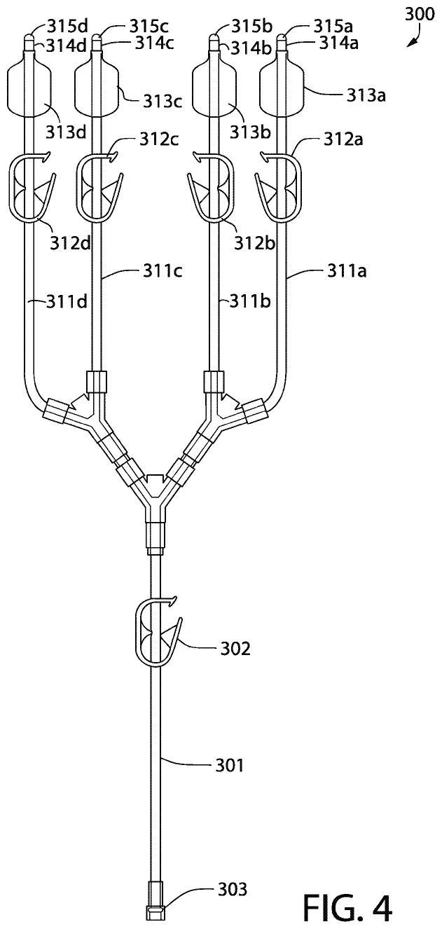

Referring to FIG. 4, the perfusion apparatus 300 will now be described. The perfusion apparatus 300 generally comprises an inlet conduit 301 and a plurality of secondary conduits 311a-d branching from the inlet conduit 301. The perfusion apparatus 300 may be made from plastic, plastic derivative, or combination thereof.

The secondary conduits 311a-d respectively comprise distal tip regions 314a-d, which includes secondary conduit outlets 315a-d. The secondary conduit outlets 315a-d allow fluid communication between the internal passageways (or lumens) of the secondary conduits 311a-d and the aortic branches of a patient when coupled thereto, such as visceral arteries. The tip portions 314a-d of the secondary conduits 311a-d may have an outer diameter that is tapered to allow easier insertion into the aortic branches of the patient. In some embodiments, the secondary conduits 311a-d respectively comprise stabilizing features 313a-d which is proximate the tip regions 314a-d. The stabilizing features 313a-d are configured to ensure a sealed fluid coupling between the passageways/lumens of the secondary conduits 311a-d and the aortic branches to which they are coupled. In some embodiments, the stabilizing feature 313 is a compressible ring-structure, such as a fluid-filled balloon.

The perfusion apparatus 300 further comprises a valve assembly that allows a user to selectably allow and/or prevent the flow of fluid, such as blood, through the inlet conduit 301 and/or secondary conduits 311a-d. In the exemplified embodiment, the valve assembly comprises a valve operably coupled to the inlet conduit 301 and a valve operably coupled to each of the secondary conduits 311a-d. In the exemplified embodiment, the valve operably coupled to the inlet conduit 301 is in the form of a clamp 302 while the valves operably coupled to each of the secondary conduits 311a-d are in the form of clamps 312a-d. While the valves are exemplified as pinch clamps 302, 312a-d, other suitable type of valves can be used, such as a globe valve, a gate valve, a ball valve, a butterfly valve, a diaphragm valve, a plug valve, a needle valve, an angle valve, or combinations thereof.

The valve assembly, formed by valves 302, 312a-d, is arranged so that it can utilized to allow or prevent flow selectably through all, none, a desired singular one, or a desired combination of the secondary conduits 311a-d. As such, in the exemplified embodiment, each of the clamps 302, 312a-d can be individual altered by a user between an open flow state (in which blood can flow through the respective secondary conduit 311a-d) and a closed seal state (in which blood cannot flow through the respective secondary conduit 311a-d). As shown in FIG. 4, all of the clamps 302, 312a-d are in the open flow state.

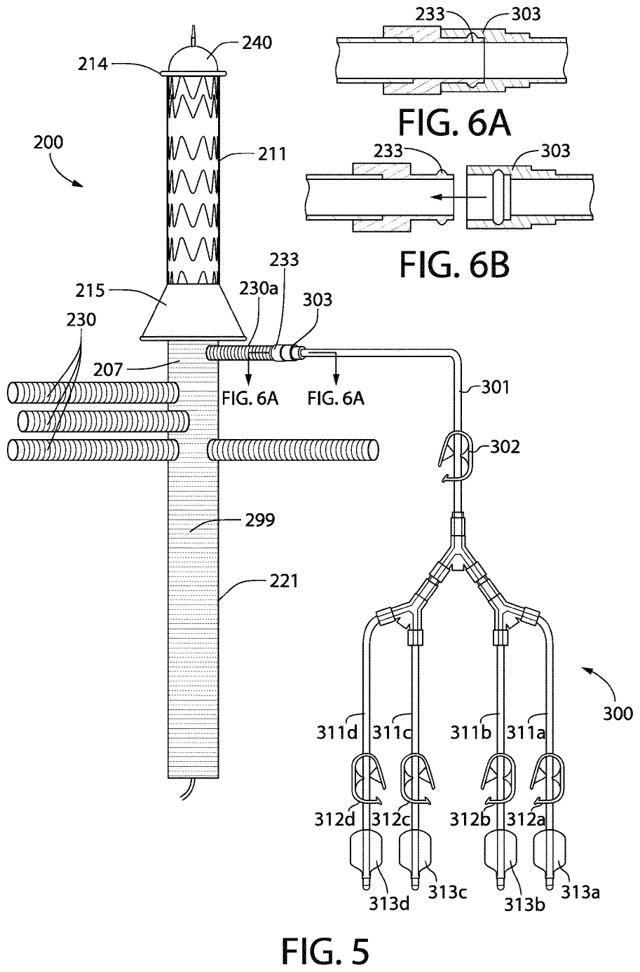

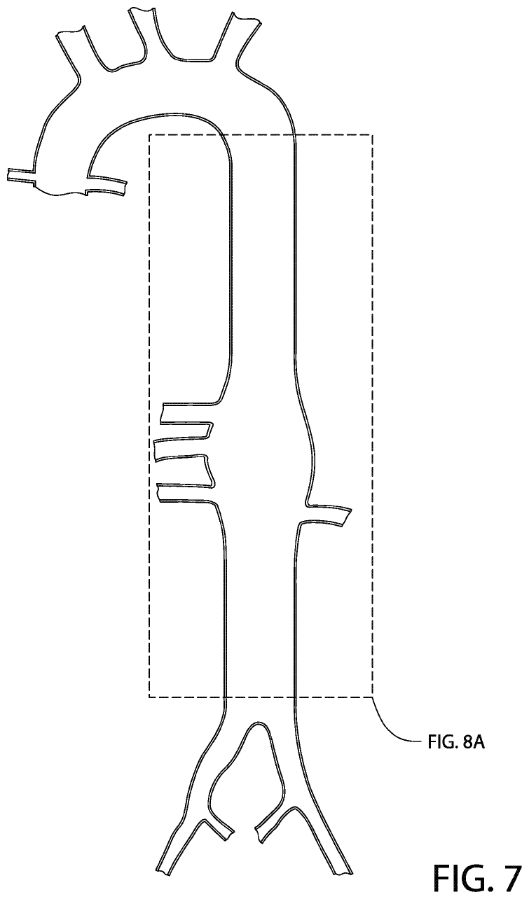

As shown in FIGS. 5 to 6B, the proximal end of the inlet conduit 301 is configured to be detachably coupled to the perfusion outlet opening 232a to form a fluid tight connection. In the exemplified embodiment, this is achieved by the presence of (and mating between) a first quick connect feature 233 on the perfusion branch conduit 230a and a second quick connect feature 303 on the inlet conduit 301. The first quick connect feature 230 may formed of material that is more rigid than the material of which the perfusion branch conduit 230a is formed, such as a plastic.

The first quick connect feature 230 may be a bayonet connector component, a threaded connector component, a snap-fit connector component, an interference fit connector component, or a combination thereof. Likewise, the second quick connect feature 303 would be the corresponding component that would mate with the selected structure of the first quick connect feature 230. Thus, the second quick connect feature 303 would take the form of the corresponding one of a bayonet connector component, a threaded connector component, a snap-fit connector component, an interference fit connector component, or a combination thereof. When mated, the quick connect features 233, 303 fluidly couple the perfusion branch lumen 231a to the passageway/lumen of the inlet conduit 301 to supply blood thereto.

It should be note that, while the perforation apparatus 300 is shown as being separate component that is coupled to the vascular prosthesis 200, in other embodiments the perforation apparatus 300 can be permanently integrated into the vascular prosthesis 200 such that it is supplied as singular component. In such an embodiment, the Referring to FIGS. 8A through 8I, the present application may comprise a method of repairing an aortic aneurysm using the above-described vascular prosthesis 200 and perfusion apparatus 300.

Referring to FIG. 8A, the vascular prosthesis 200 is inserted into the aorta of a patient. In some embodiments, the prosthesis 200 is inserted into the descending thoracoabdominal aorta. In a some embodiments, the prosthesis 200 is inserted into the descending thoracoabdominal aorta below the visceral arteries. As shown in FIGS. 8B and 8C, the sheath 208 surrounding the vascular prosthesis is removed and the prosthesis 200 is expanded so that the outer surface 205 of the vascular prosthesis 200 is in contact with the walls of the aorta. A molding balloon 240 is inflated to spatially stabilize the proximal end of the vascular prosthesis 200 against the walls of the aorta.

Referring to FIG. 8D, the prosthesis 200 may then be fluidly coupled to the aorta by attaching the hemostatic apron 215 to the walls of the aorta. In some embodiments, the hemostatic apron 215 is attached to the walls of the aorta by sutures. In some embodiments, a clamp is placed in the clamping zone 207 to prevent the flow of fluid through the primary lumen 201 of the prosthesis 200. As shown in FIG. 8E, the portion of the aorta below the hemostatic apron 215 may then be opened to expose the rest of the prosthesis 200.

Referring to FIG. 8F, the inlet conduit 301 of the perfusion apparatus 300 may then be fluidly coupled to the distal end of a perfusion branch conduit 230a. The valve assembly (302, 312) of the perfusion apparatus 300 is in a closed sealed state so that fluid such as blood from the primary lumen 201 of the vascular prosthesis 200 cannot flow through the plurality of secondary conduits 311 of the perfusion apparatus 300 that branch from the inlet conduit 301.

Referring to FIGS. 8G and 8H, after the perfusion apparatus 300 is connected to the vascular prosthesis 200, one or more secondary conduits 311 may then be fluidly coupled to one or more aortic branches. In some embodiments, the secondary conduit is fluidly connected to an aortic branch by having one or more tip portions be inserted into the proximal end of one or more aortic branches, and then attached with a soft clamp. The soft clamp would be tight enough to still allow fluid to pass through to still allow fluid communication but would prevent the stabilizing feature from passing through. Once the secondary conduit is fluidly coupled to an aortic branch, the valve assembly (302, 312) is altered to an open flow state to restore blood flow to one or more aortic branches.

As shown in FIG. 8I, the distal end of the vascular prosthesis 200 may then be cut to an appropriate length that would be known by a surgeon of skill in the art, thereby forming a new distal end of the vascular prosthesis 200. As shown in, FIG. 8J, the new distal end of the vascular prosthesis 200 is then attached to a downstream portion of the aorta. In some embodiments, the new distal end of the vascular prosthesis 200 is attached to the downstream portion of the aorta with sutures.

Referring to FIGS. 8J through 8M, the clamp 110 is removed from the clamping zone 207 and the one or more secondary conduits 311 are then removed from the one or more aortic branches. One or more branch conduits 230 of the vascular prosthesis 200 are then attached to one or more aortic branches. In certain embodiments, a clamp is placed at the end of each of the one or more branch conduits 230 of the vascular prosthesis 200to prevent the flow of fluid through the branch lumen 231. When the one or more branch conduits 230 of the vascular prosthesis 200 are attached to one or more aortic branches, the clamp 110 is then removed to restore blood flow.

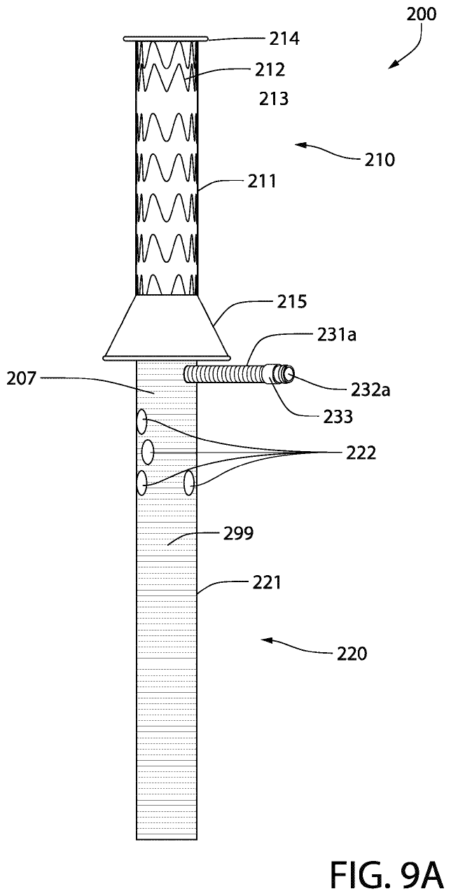

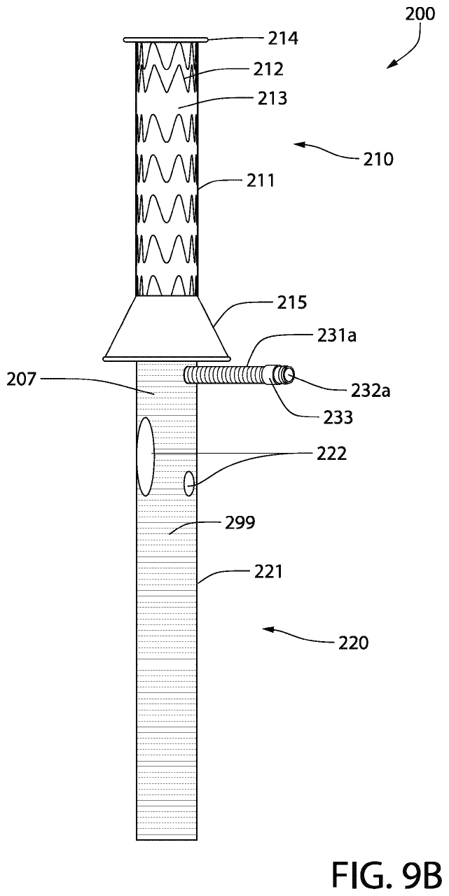

Referring now to FIGS. 9A and 9B, the vascular prosthesis 200 may be configured in a variety of alternative configurations including some embodiments without any branch conduits 230. In some embodiments, the openings 222 can be fluidly coupled directly to an aortic branch such as by using a Carrel patch. In such embodiments, one or more of the branch conduits 230 can be omitted while the associated opening 222 remains such that the opening can be directly connected to an aortic branch by using the Carrel patch. As shown in FIG. 9B, the openings 222 may be of various sizes. In such embodiments, multiple aortic branches may be directly connected to a single opening 222.

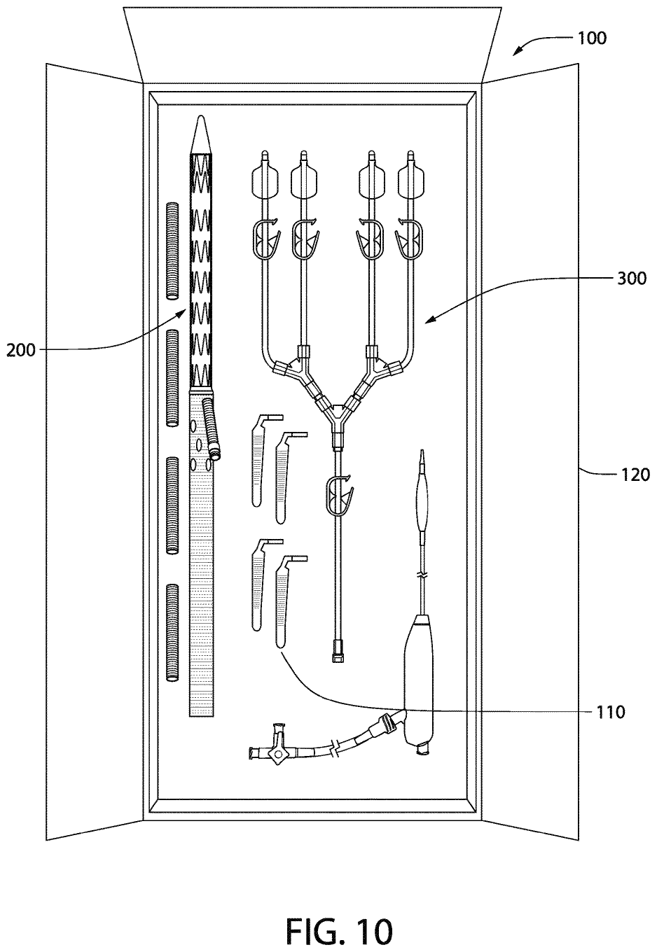

As shown in FIG. 10, the surgical kit 100 may comprise a plurality of branch conduits 230 that are detached from the vascular prosthesis. The branch conduits 230 may then be attached during surgery, as needed. In some embodiments, a plurality of aortic branches may be coupled to the vascular prosthesis using any combination of direct coupling with an opening 222 and coupling with a branch conduit 230.

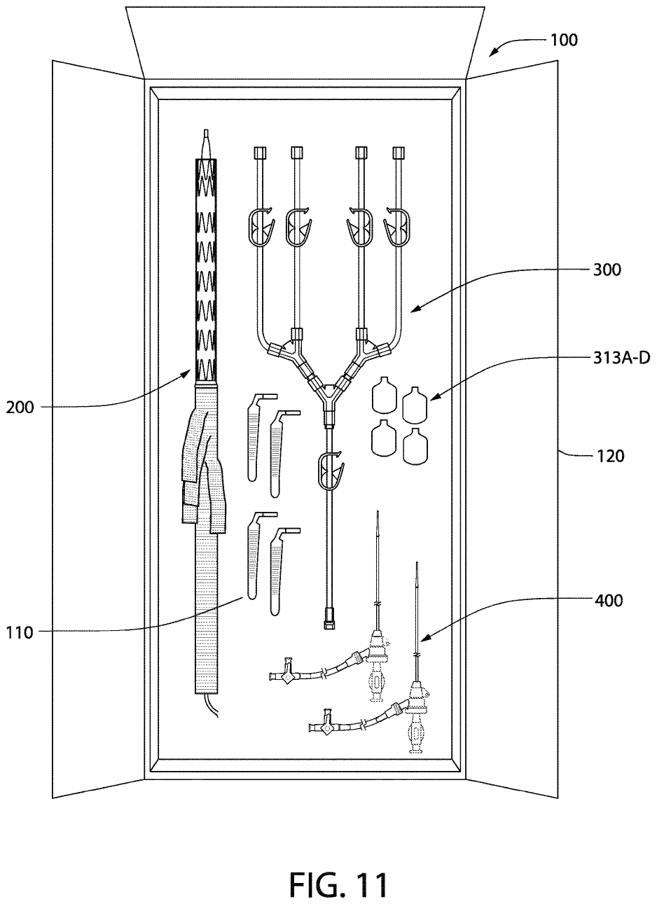

Referring to FIG. 11, a surgical kit 100 is disclosed according to some embodiments. The surgical kit 100 is similar to the surgical kit 100 described above, and generally comprises a vascular prosthesis 200, a perfusion apparatus 300, one or more introducer sheath 400, and at least one surgical accessory 110. There may be any number of introducer sheaths 400 in the surgical kit 200. In some embodiments, there is between one and five introducer sheaths 400. The vascular prosthesis of the alternative embodiment is generally the same as the vascular prosthesis 200 described above, but may lack a profusion branch conduit 230a. The perfusion apparatus 200 may be fluidly coupled to the aorta of a patient through the introducer sheath 400, through the perfusion branch conduit 230a of the vascular apparatus 200, or any combination thereof.

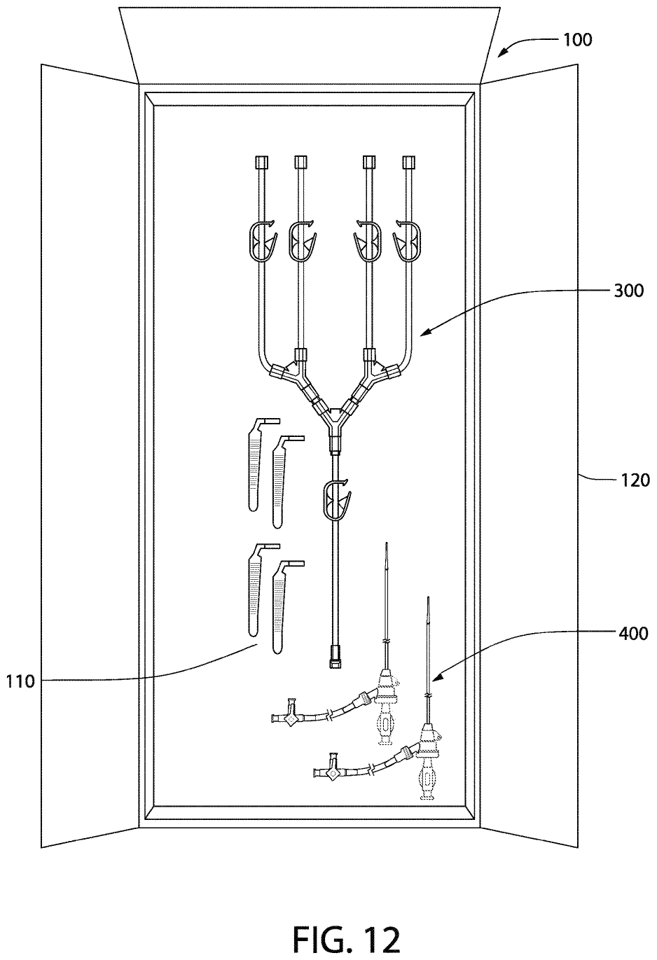

Referring to FIG. 12, a surgical kit 100 is disclosed according to some embodiments. The surgical kit 100 is similar to the surgical kits 100 described above and generally comprises a perfusion apparatus 300, one or more introducer sheath 400, and at least one surgical accessory 110.

The introducer sheath 400 is generally known in the art, such as those disclosed in U.S. Pat. Nos. 9,597,063; 9,597,480; 10,398,469; and 11,672,564. The disclosures of the aforementioned patents are incorporated herein by reference. In some embodiments, the introducer sheath 400 may comprise a sheath, guidewire, entry needle, and tubing. The components may be formed from any material known in the art including nitinol, stainless steel, plastic, or plastic derivative. In some embodiments, the introducer sheath 400 may comprise a flexible tube and a connection component. The connection component may be selected from any known in the art. In some embodiments, the connection component is selected from a group consisting of bayonet connector component, a threaded connector component, a snap-fit connector component; an interference fit connector component, and combinations thereof. In some embodiments, the one or more introducer sheath may further comprise a valve assembly that allows a user to selectably allow and/or prevent the flow of fluid, such as blood, to flow through the flexible tube.

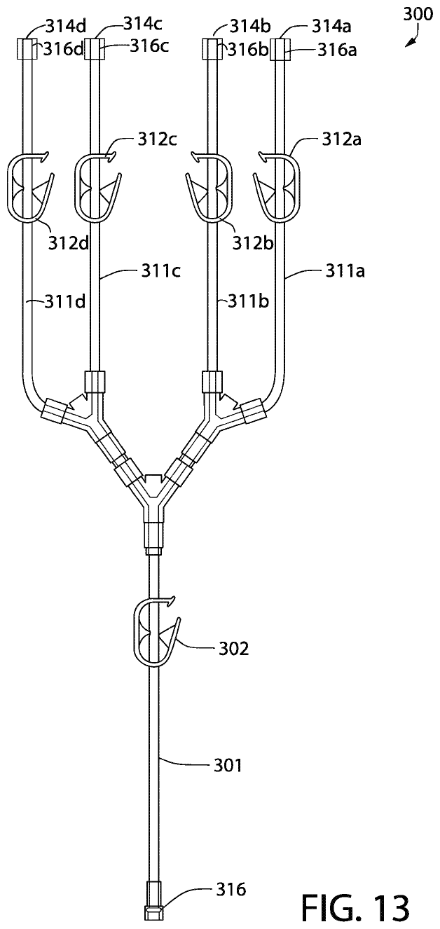

As shown in FIG. 13, a perfusion apparatus 300 is illustrated according to some embodiments. The perfusion apparatus 300 may comprise an inlet conduit 301 and a plurality of secondary conduits 311a-d branching from the inlet conduit 301. The perfusion apparatus 300 may be made from plastic, plastic derivative, or combination thereof.

The inlet conduit 301 may comprise a connection feature 316. The connection feature 316 may be any connection feature known in the art and is configured to be coupled to the connection component of the flexible tubing of the one or more introducer sheath 400. The connection component 316 may be any connection structure known in the art. In some embodiments, the connection component is selected from a group consisting of a bayonet connector component, a threaded connector component, a snap-fit connector component; an interference fit connector component, and combinations thereof. The connection feature 316 may be configured to be mateable with the corresponding connection component of the flexible tubing to fluidly couple the perfusion apparatus 300 to the one or more introducer sheath 400.

The secondary conduits 311a-d respectively comprise distal tip regions 314A-d, which comprise secondary connection features 316a-d. The secondary connection features 316a-d are configured to mate with the corresponding connection components of the respective introducer sheath 400. The secondary connection features 316a-d are the same as the connection feature 316, and allows fluid communication between the internal passageways (or lumens) of the secondary conduits 311a-d and the aortic branches of a patient when coupled thereto, such as visceral arteries.

As shown in FIG. 11, the surgical kit 100 may include stabilizing features 313a-d which are configured to ensure a sealed fluid coupling between the passageways/lumens of the secondary conduits 311a-d and the aortic branches to which they are coupled. In some embodiments, the stabilizing feature 313 is a compressible ring-structure, such as a fluid-filled balloon.

The perfusion port can be connected to an introducer sheath. The introducer sheath is then inserted in an area before the stent-graft site. The needle is removed and the quick connect feature of the perfusion port is connected to the corresponding connect feature of the introducer sheath. The secondary connections are then connected to secondary arteries, as needed. Same procedure, inserting introducer sheath, remove needle, insert corresponding line. The lines can come in a kit. The lines can be connected or detached. There lines may be attached to a third portion

The present application may comprise an alternative method of repairing an aortic aneurysm using the alternative surgical kit 100 comprising the vascular prosthesis 200, the perfusion apparatus 300, and introducer sheath 400.

The introducer sheath 400 may be inserted into the aorta of a patient at a site that is closer to the heart than the portion of aorta that needs to be repaired. The guide wire and needle of the one or more introducer sheath 400 are then removed, leaving a flexible tube with a connection component. The connection component of the one or more introducer sheath 400 is then coupled to the connection feature 316 of the inlet conduit 301 of the perfusion apparatus 300. In some embodiments, a second introducer sheath 400 is inserted into the one or more aortic branches, the guidewire and needle of the second introducer sheath 400 are then removed, leaving a second flexible tube with a connection component. The connection component of the second flexible tube is then coupled to one of the connection features 316a-d of the secondary conduits 311a-d of the perfusion apparatus 300. This procedure may then be replicated for any required aortic branch, with each aortic branch being coupled to one of the secondary conduits. After the inlet and secondary conduits 301, 311a-d are coupled to the one or more introducer sheath 400 and second introducer sheath 400, any necessary valves are altered to an open flow state to allow blood to flow from the aorta, through the perfusion apparatus, to the one or more aortic branches.

After the perfusion apparatus 300 is connected to the aorta and the one or more aortic branch conduits, the aorta may be clamped at a site below the one or more introducer sheath 400, and the vascular prosthesis is inserted into the aorta of a patient and deployed in a manner similar to the surgical method described above in relation to FIGS. 8A-M, except without needing to couple the vascular prosthesis 200 to the perfusion apparatus 300. After the vascular prosthesis 200 is deployed and attached to the aorta and the one or more aortic branches, the one or more introducer sheaths 400 may be removed, and the site of insertion may be repaired using any means known in the art.

Some embodiments herein relate to redirecting blood flow from an inflow artery to one or more aortic branch. In some embodiments, the inflow artery is an aorta, a subclavian artery, or an axillary artery. In a configuration, the flow of blood into an aortic branch may be about 0.8 to 1 L/minute. The size of a conduit herein may be any suitable for redirecting the blood flow. In a configuration, a conduit herein has a size of 8-12 Fr.

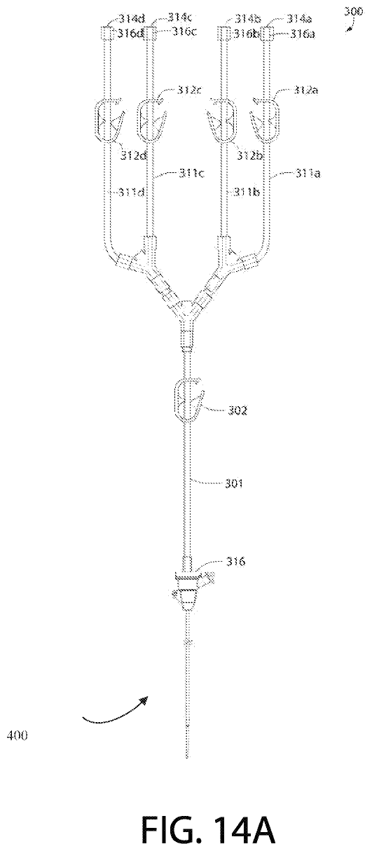

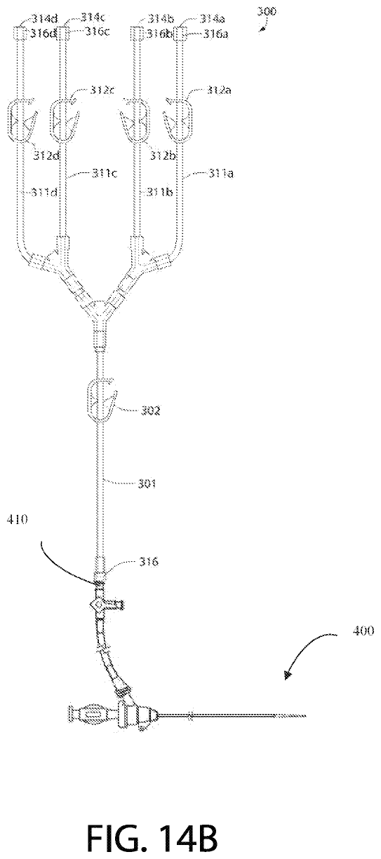

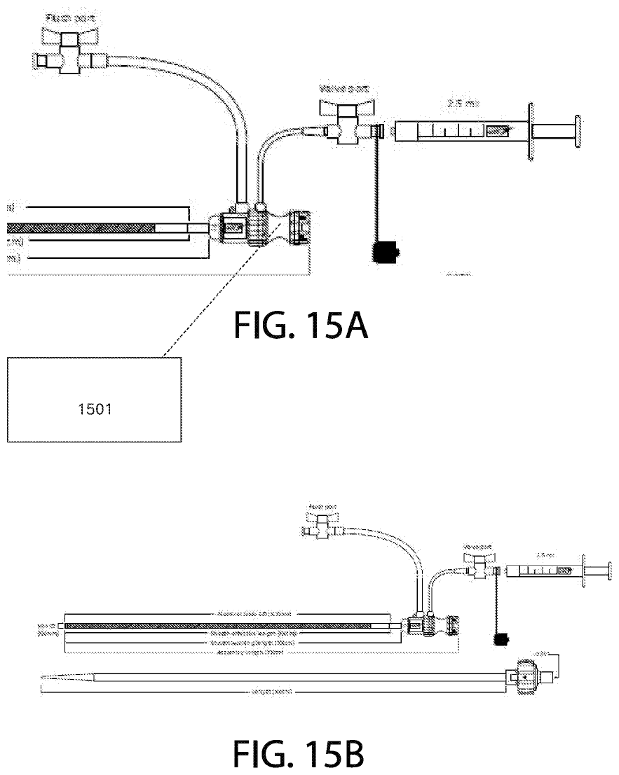

Some embodiments herein comprise one or more introducer sheath apparatus. In some embodiments, an introducer sheath is connected or connectable to a perfusion apparatus at the proximal end of the inlet conduit thereof. Examples of connections between the introducer sheath apparatus 400 and the perfusion apparatus 300 are illustrated in FIGS. 14A and 14B. As illustrated in FIG. 14A, the proximal end of the inlet conduit 301 is engaged with a port on the introducer sheath 400. A portion of the introducer sheath 400 is not illustrated in FIG. 14A but one of skill in the art would recognize that the accessory tubing an port illustrated in FIGS. 11 and 14B may be present in the embodiment of FIG. 14A. FIG. 14B illustrates connecting the proximal end of the inlet conduit 301 to an accessory port of the introducer sheath 400. In some embodiments of, an introducer sheath apparatus comprises a valve configured to selectively prevent flow through the introducer sheath. The valve may be any known in the art. In some embodiments, the valve is normally closed such that when a dialator or needle is withdrawn, the valve closes to prevent flow of blood out of the introducer sheath. In some embodiments, the valve comprises an inflatable balloon having a lumen space that closes upon inflation, thus closing the valve. A non-limiting example of a valve in an introducer sheath is that in a GORE® DRYSEAL Flex Introducer. A non-limiting example of an introducer sheath apparatus herein is the GORE® DRYSEAL Flex Introducer. See https://www.goremedical.com/products/dryseal. See also Jones, M. et al. “Use of the GORE® DrySeal Flex Introducer Sheath to Facilitate Implantation of the Transcatheter Venus P-valve” Congenital Heart Disease, 16(3): 197-203, which is incorporated by reference as if fully set forth. See also FIGS. 15A and 15B, which illustrate the GORE® DRYSEAL Flex Introducer. A valve 1501 located within the valve closes when the port is not forced open by a dilator. However, the valve can be engaged by an actuating portion of the proximal end of an inlet conduit when the proximal end is engaged with the port, and thereby open the valve to permit flow of blood out of the introducer sheath. The proximal end of an inlet conduit may comprise a protruding collar that when inserted into the port opens the valve. An inlet conduit of a perfusion apparatus herein may be connected to any of the three ports illustrated in FIGS. 15A and 15B. An inlet conduit of a perfusion apparatus herein may be connected to the port comprising valve 1501.

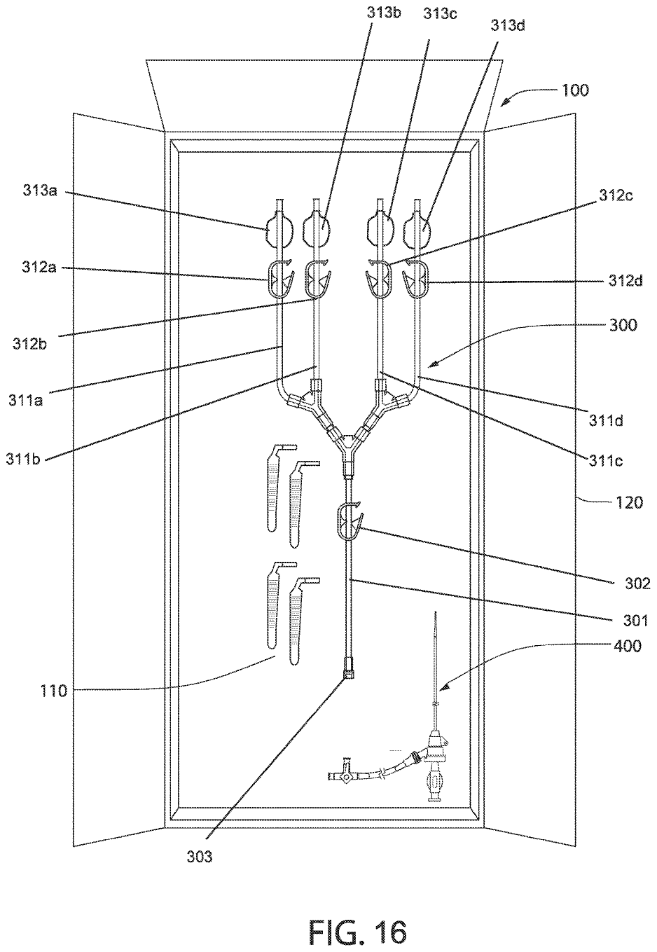

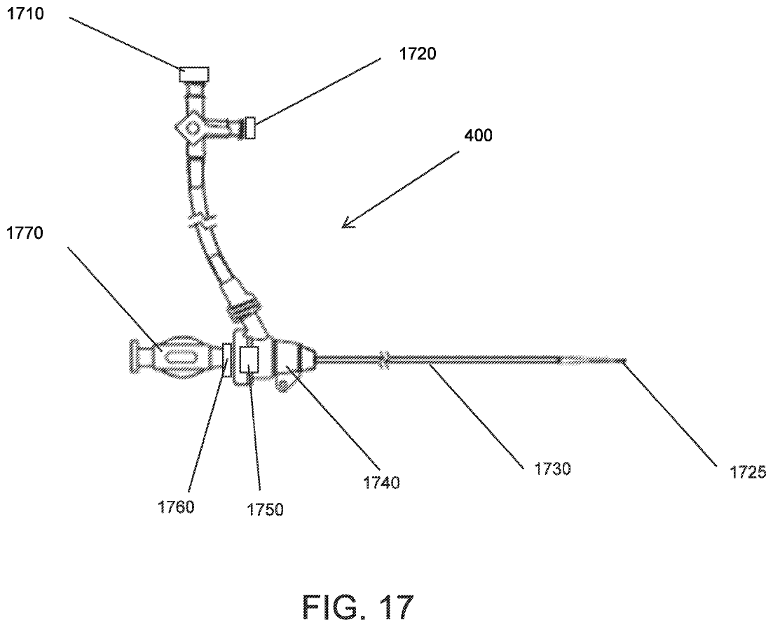

Referring to FIG. 16, an embodiment is illustrated comprising a perfusion apparatus 300 and an introducer sheath apparatus 400. The embodiment illustrated also optionally comprises surgical accessories 110. As illustrated, the embodiment is a surgical kit 100. The skilled artisan will recognize, however, that the components illustrated may also comprise a system or all or a portion of a medical device herein. The perfusion apparatus 310 comprises stabilizing features 313a-d, clamps 312a-d, secondary conduits 311a-d, and a connecting feature 303. In an embodiment, the stabilizing features are removable from the secondary conduits to which they are attached. In some embodiments the stabilizing features permanently affixed to the secondary conduits to which they are attached. In some embodiments the stabilizing features are integral with secondary conduits to which they are attached. In an embodiment, the stabilizing features are compressible rings. In an embodiment, the stabilizing features are balloons. In an embodiment, the stabilizing features are fluid filled balloons. The stabilizing features may be independently selected from options for a stabilizing feature. As illustrated, the stabilizing features 312a-d are fluid-filled balloons. The connection feature 303 is configures to engage with a port of the introducer sheath apparatus 400. Non-limiting embodiments an introducer sheath apparatus, a perfusion apparatus, and engagement between a connection feature of a perfusion apparatus to a port of the introducer sheath apparatus 400 are described below with reference to FIGS. 17 to 21.

As illustrated in FIG. 16, a valve assembly is provided comprising clamps 312a-d. The valve assembly is configured to selectably alter the plurality of secondary conduits between an open flow state and a closed sealed state. In an embodiment, one or more of the valves in a valve assembly herein is mounted on a secondary conduit such that it remains mounted when the secondary conduit is in the open or closed state. As illustrated in FIG. 16, each of clamps 311a-d are mounted to the respective secondary conduits 311a-d. The embodiment illustrated comprises a pinch clamp for each of clamps 311a-d. Each may comprise one or more through holes (not illustrated) through which the respective secondary conduit runs and thereby remain mounted whether the pinch claim is open or closed to alter the secondary conduit between the open state or the closed state. A valve assembly may also comprise a valve to alter the inlet conduit between an open and a closed state. As illustrated in FIG. 16, a clamp 302 is mounted on the inlet conduit 301 such that the inlet conduit can be altered between the open state and the closed state and thereby regulate flow into the secondary conduits 311a-d. Like clamps 311a-d, clamp 302 is illustrated as a pinch clamp and may also be mounted such that it remains mounted when the inlet conduit is in either the open or the closed state.

An introducer sheath apparatus may be configured to comprise a port to fluidly connect to a proximal end of an inlet conduit of a perfusion apparatus. The port may be a dilator port or one or more secondary ports. A secondary port may be an infusion port or the like as known in the art. Referring to FIG. 17, an embodiment of an introducer sheath 400 is illustrated and comprises a first secondary port 1710, a second secondary port 1720, a sheath tube 1730, a hub 1740, a dilator valve 1750, a dilator port 1760, and a gripping portion 1770 of the dilator 1725. The sheath tube 1730 extends from the hub 1740. The dilator 1725 may be utilized during insertion of the introducer sheath into an artery. After insertion, dilator 1725 may be retracted. A perfusion apparatus may be connected to any one of the first secondary port 1710, the second secondary port 1720, or the dilator port 1760.

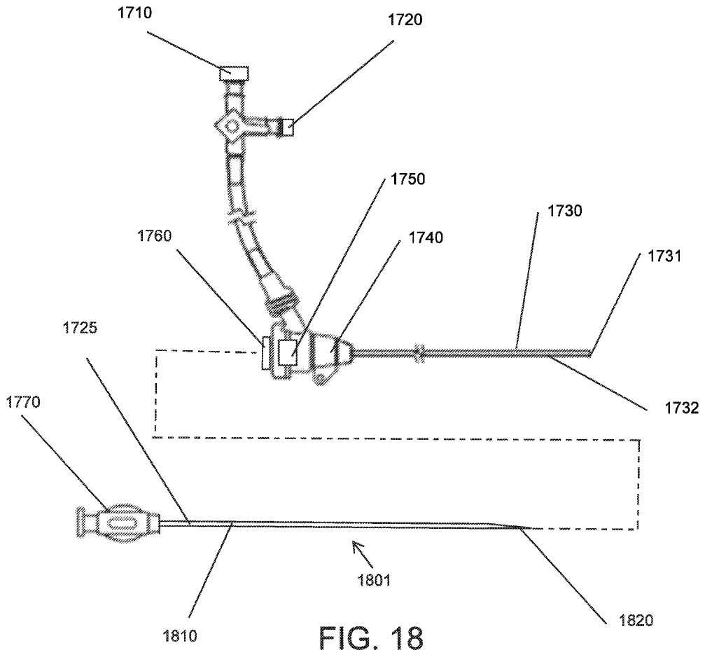

Referring to FIG. 18, the introducer sheath apparatus 400 of FIG. 17 is illustrated with the dilator 1725 in a retracted state. Collectively, the gripping portion 1770 and the dilator 1725 may be referred to as in introducer 1801. As illustrated, the introducer 1801 also comprises a shaft portion 1810 and a tapered tip portion 1820. The introducer 1801 is alterable from: (1) an inserted state in which the tapered tip portion protrudes from a distal end 1731 of the sheath tube 1730, and the shaft portion 1810 is located within a sheath lumen 1731, which may also be referred to as a working lumen; and (2) a retracted state in which the shaft portion 1810 of the introducer 1801 is removed from the sheath lumen 1732 such that blood can flow through the sheath lumen to the dilator port 1760. FIG. 17 illustrates the inserted state while FIG. 18 illustrates the retracted state with a dashed line to illustrate the pathway of retracting or inserting the introducer 1801. In an embodiment, an introducer also comprises a guidewire (not illustrated).

The dilator valve 1750 in an embodiment is alterable between: (1) an open state in which blood can flow from the sheath lumen 1732 to the dilator port 1760; and (2) a closed state in which blood is prohibited form flowing from the sheath lumen 1732 the dilator port 1760.

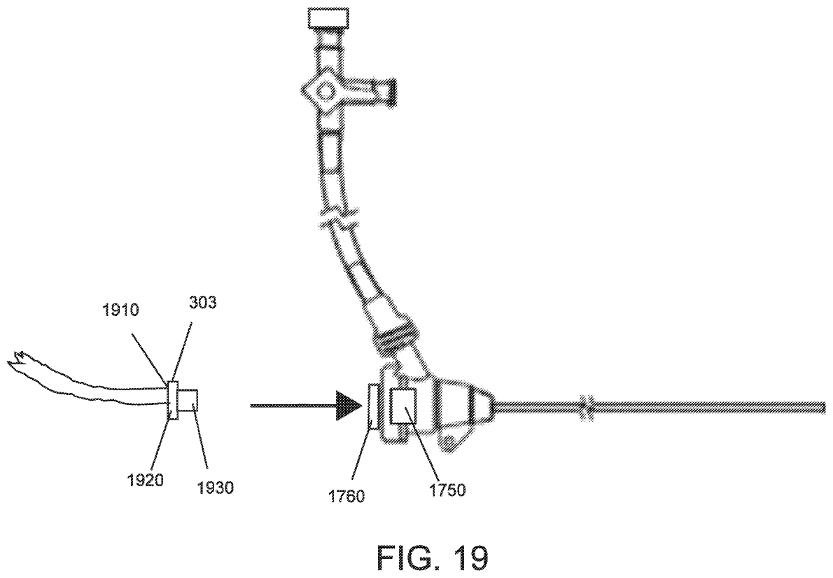

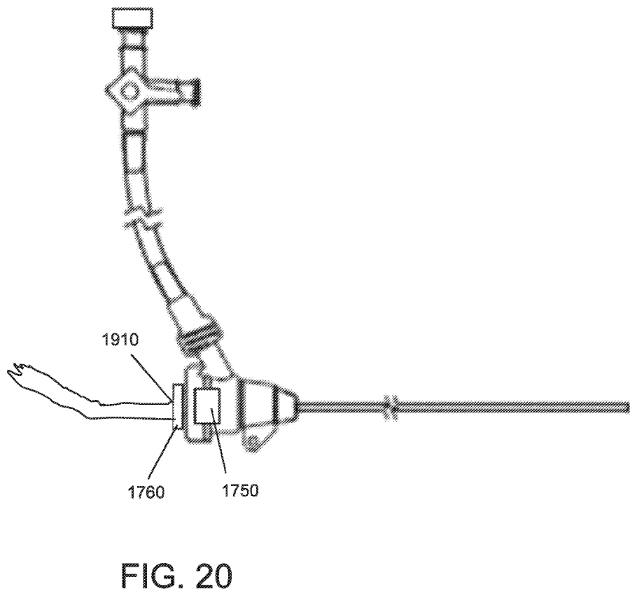

Referring to FIG. 19, a proximal end 1910 of an inlet conduit of a perfusion apparatus is illustrated. The proximal end 1910 comprises a connecting feature 303. A connecting feature of an inlet conduit may also be referred to as a primary connecting feature. In the embodiment illustrated in FIG. 19, the connecting feature 303 comprises a collar 1920 and an insert portion 1930. As illustrated, the connecting feature is not yet engaged with the dilator port 1760. However, as indicated by the arrow, the proximal end 1901 may be engaged with the dilator port 1760. During engagement, the insert portion 1930 may actuate the valve 1750 to place the valve in the open state such the blood may flow into the inlet conduit. FIG. 20 illustrates the connecting feature 303 engaged with the dilator port 1760. In such a configuration, the introducer sheath apparatus is fluidly coupled to the inlet conduit of the perfusion apparatus to deliver blood from the aorta or another inflow artery to the inlet conduit.

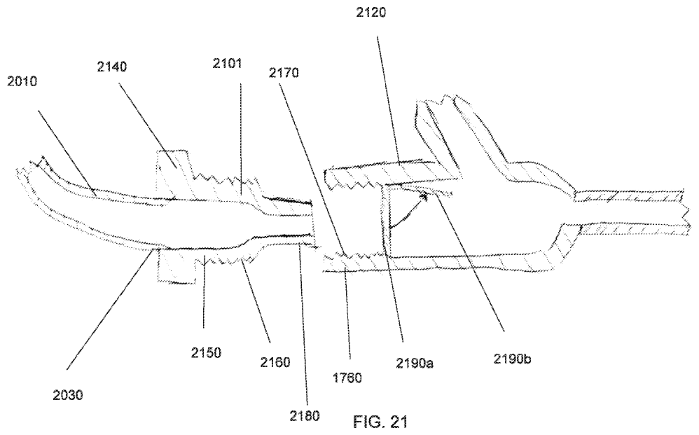

FIG. 21 illustrates an embodiment of a primary connection feature 2101 of an inlet conduit 2010 and a port connection feature 2120 of a dilator port 1760 of an introducer sheath apparatus in cross section. The dilator port 1760 of the introducer sheath apparatus comprises the port connection feature 2020 for coupling to a primary connection feature 2101 of the proximal end 2030 of the inlet conduit 2010. The primary connection feature 2101 comprise a collar 2140, an insert 2105, and a tip 2180. The insert 2150 comprises ridges 2160 configured to mate with ridges 2170 within the dilator port 1760. As illustrated, the tip 2180 upon insertion of into the dilator port 1760 engages with a dilator valve, which is illustrated as 2190a in a closed state and 2109b in an open state. Fluid pressure of blood within the introducer sheath tube may maintain the valve in the closed state. However, insertion of the tip 2180 actuates the valve to the open state such that the inlet conduit 2010 and the introducer sheath tube are fluidly coupled. In an embodiment, the ridges are snap fit. In an embodiment the ridges represent rings of a screw fitting. The skilled artisan will recognize that different configurations of the primary connection feature and port connection feature may be implemented in embodiments herein. Further, the connection features as described for the primary connection feature and the port connection feature of the dilator port may be similarly implanted on one or more of the secondary ports. The secondary port connection features may be the same or different than those of the dilator port connection feature.

Some embodiments of a system, method, medical device, or kit herein comprise a valve assembly. Each valve of a valve assembly may be the same. A valve assembly may comprise more than one type of valve. Non-limiting examples of valve that can be selected for a valve assembly comprise globe valves, gate valves, ball valves, butterfly valves, diaphragm valves, plug valves, needle valves, and angle valves.

Some embodiments of a system, method, medical device, or kit herein comprise a perfusion apparatus comprising secondary conduits, each with a distal end. In some embodiments one or more or all of the distal ends comprise a balloon fixed thereto. In some embodiments, the balloon configured to fit tightly enough in the respective aortic branch in which it is configured to be inserted such that the distal end is held fast during a method herein. In some embodiments, the balloon is integral with the distal ends of the secondary conduits. As mentioned, FIG. 11 illustrates stabilizing features 313a-d proximate the tip regions 314a-d. The stabilizing features 313a-d are configured to ensure a sealed fluid coupling between the passageways/lumens of the secondary conduits 311a-d and the aortic branches to which they are coupled. In some embodiments, the stabilizing feature 313 is a compressible ring-structure, such as a fluid-filled balloon. In some embodiments, stabilizing features as illustrated in FIG. 11 are separate from the secondary conduit tip regions 314a-d and configured to be fixed thereto in use. In some embodiments, the stabilizing features are permanently fixed to tip regions 314a-d. In some embodiments, the stabilizing features are integral with tip regions 314a-d. In some embodiments, a stabilizing feature are permanently fixed or integral with a distal end of a secondary conduit herein is a compressible ring structure. In some embodiments, a stabilizing feature that is permanently fixed or integral with a distal end of a secondary conduit herein is a balloon. In some embodiments, a stabilizing feature that is permanently fixed or integral with a distal end of a secondary conduit herein is a fluid-filled balloon.

Some embodiments of a system, method, medical device, or kit herein comprise a perfusion apparatus and a vascular prosthesis comprising a perfusion conduit, where the perfusion apparatus connects to the perfusion conduit. In some embodiments, the port connection feature, connecting features connecting a perfusion apparatus to the port of an introducer sheath apparatus, and the connection features to connect to a perfusion conduit of a prosthesis are the same.

In some embodiment all of the distal ends on secondary conduits of a perfusion apparatus herein comprise a stabilizing feature. In some embodiment all of the distal ends on secondary conduits of a perfusion apparatus herein comprise a stabilizing feature fixed thereto. In some embodiment all of the distal ends on secondary conduits of a perfusion apparatus herein comprise a stabilizing feature permanently fixed thereto. In some embodiment all of the distal ends on secondary conduits of a perfusion apparatus herein comprise a stabilizing feature integral therewith. In some embodiments, the stabilizing feature is a compressible ring structure. In some embodiments, the stabilizing feature is a balloon. In some embodiments, the stabilizing feature is a fluid-filled balloon.

Some embodiments comprise a system, method, medical device, or kit. In some embodiments, a perfusion apparatus and a introducer sheath apparatus in a system, method, medical device, or kit herein are separate pieces configured to be connected to one another. In some embodiments, a perfusion apparatus and a introducer sheath apparatus in a system, method, or medical device herein are separable but connected to one another. In some embodiments, a perfusion apparatus and a introducer sheath apparatus in a system, method, or medical device herein are connected to one another. In some embodiments, a perfusion apparatus and a introducer sheath apparatus in a system, method, or medical device herein are permanently connected to one another. In some embodiments, a connection between a perfusion apparatus and a introducer sheath apparatus herein is releasable. In some embodiments, a connection between a perfusion apparatus and a introducer sheath apparatus herein is permanent. In some embodiments, a connection between a perfusion apparatus and a introducer sheath apparatus herein is an integral connection.

Some embodiments comprise a system, method, medical device, or kit. In some embodiments, the system, method, medical device, or kit comprises a perfusion apparatus, a valve assembly, and an introducer sheath apparatus. In some embodiments, the perfusion apparatus is chosen from one described herein. In some embodiments, the valve assembly is chosen from one described herein. In some embodiments, the introducer sheath apparatus is chosen from one described herein. In some embodiments, the introducer sheath apparatus is chosen from one described U.S. Pat. Nos. 9,597,063; 9,597,480; 10,398,469; and 11,672,564, all of which are incorporated herein by reference as if fully set forth.

Some embodiments comprise a system comprising a perfusion apparatus, a valve assembly, and an introducer sheath apparatus. In some embodiments, perfusion apparatus comprises an inlet conduit comprising a proximal end, and a plurality of secondary conduits branching from the inlet conduit and configured for fluid coupling to aortic branches. In some embodiments, the valve assembly configured to selectably alter the plurality of secondary conduits between an open flow state and a closed sealed state.

In some embodiments, the introducer sheath apparatus is configured to be fluidly coupled to the inlet conduit of the perfusion apparatus to deliver blood from the aorta or another inflow artery to the inlet conduit. In some embodiments, the other inflow artery is selected from a subclavian artery or axillary artery. In some embodiments, the introducer sheath apparatus comprises: a sheath tube configured for insertion into the aorta or other inflow artery, the sheath tube forming a working lumen. In some embodiments, the introducer sheath comprises a port in fluid cooperation with the sheath lumen, where the port is configured to be fluidly coupled to the proximal end of the inlet conduit of the perfusion apparatus to fluidly couple the working lumen to the inlet conduit. In some embodiments, the introducer sheath apparatus further comprises a valve configured to selectively prevent flow through the introducer sheath apparatus to the inlet conduit.

In some embodiments, the introducer sheath apparatus further comprises one or more of a dilator and a guide wire. The dilator may be a needle. In some embodiments, the port of the introducer sheath apparatus comprises a port connection feature for coupling to a primary connection feature of the proximal end of the inlet conduit.

In some embodiments, each of the plurality of secondary conduits of the perfusion apparatus has a distal end configured to be fluidly coupled to one of the aortic branches.

In some embodiments, the distal ends of the plurality of secondary conduits comprises a tip portion having a tapered outer diameter.

In some embodiments, the system further comprises a balloon fixed to the distal end(s) of one or more or each of the plurality of secondary conduits. In some embodiments, the balloon configured to tight fit in the respective aortic branch in which it is configured to be inserted.

Some embodiments of the system further comprise a plurality of the introducer sheath apparatuses. The port of each of the plurality of the introducer sheath apparatuses, in some embodiments, comprises a port connection feature. The proximal end of the inlet port, in some embodiments, comprises a primary connection feature configured to be fluidly coupled with the port connection feature of one of the plurality of the introducer sheath apparatuses. In some embodiments one or more or each of the plurality of secondary conduits of the perfusion apparatus have a distal end comprising a second connection feature configured to be connected to one of the plurality of the introducer sheath apparatuses.

In some embodiments, the primary connection feature and the second connection features are the same such that the primary connection feature can be fluidly coupled to any one of the port connection features of the plurality of the introducer sheath apparatuses and the second connection features can be fluidly coupled to any one of the port connection features of the plurality of the introducer sheath apparatuses.

In some embodiments, the primary connection feature is different from the second connection features such that the primary connection feature and the secondary connection features cannot be fluidly coupled to the same port connection features of the plurality of the introducer sheath apparatuses.

In some embodiments, the port connection feature of at least one of the plurality of the introducer sheath apparatuses is configured to be fluidly couplable to the primary connection feature but not fluidly couplable to the secondary connection features. In some embodiments, the port connection feature of the remaining plurality of the introducer sheath apparatuses are configured to be fluidly couplable to the secondary connection features but not fluidly couplable to the primary connection feature.

In some embodiments, the primary connection feature is at least one of: (i) a different size than the secondary connection features; (ii) a different type than the secondary connection feature; and (iii) has a different structure than the secondary connection features.

The primary connection feature and/or one or more of the secondary connection features may be selected from any connection feature known in the art. Non-limiting examples of connection features comprise the connection component is selected from a group consisting of bayonet connector component, a threaded connector component, a snap-fit connector component, and an interference fit connector component. The primary connection feature and/or one or more of the secondary connection features may be selected from the foregoing non-limiting examples.

In some embodiments, at least one of the plurality of the introducer sheath apparatuses is configured to allow a greater volume of blood flow therethrough at the same pressure than other ones of the plurality of the introducer sheath apparatuses.

In some embodiments, the perfusion apparatus comprises four of the secondary conduits.

In some embodiments, the valve assembly is configured to individually alter each one of the plurality of secondary conduits between the open flow state and the closed sealed state independent of the other ones of the secondary conduits. In some embodiments, the valve assembly comprises a plurality of valves, each of the plurality of valves operably coupled to one of the plurality of secondary conduits. In some embodiments, each of plurality of valves is a clamp. A valve of a valve assembly herein may be selected from any known in the art. Non-limiting examples of types of valves that may be included in a valve assembly comprise pinch clamps, globe valves, gate valves, ball valves, butterfly valves, diaphragm valves, plug valves, needle valves, and angle valves. A valve assembly herein may comprise valves all of the same type or valves of two or more different types.

In some embodiments, the system further comprises one or more a sealed packages containing the perfusion apparatus and the introducer sheath.

In some embodiments, the introducer sheath apparatus further comprises an introducer comprising a shaft portion and a tapered tip portion. The introducer, in some embodiments, is alterable from: (1) an inserted state in which the tapered tip portion protrudes from a distal end of a sheath tube of the introducer sheath apparatus and the shaft portion is located within a sheath lumen of the sheath tube for penetration of a wall of the aorta or of a wall of the other inflow artery to introduce the sheath tube into the aorta; and (2) a retracted state in which the shaft portion of the introducer is removed from the sheath lumen such that blood can flow through the sheath lumen.

In some embodiments, the system further comprises a vascular prosthesis. The vascular prosthesis may be chosen form any herein. The vascular prosthesis, in some embodiments, is free of a perfusion branch conduit. The vascular prosthesis, in some embodiments, comprises a conduit forming a primary lumen extending from a primary inlet opening to a primary outlet opening. The primary conduit can be configured to be fluidly coupled to the aorta of the patient.

In some embodiments, the vascular prosthesis further comprises a stent-graft portion, a tube-graft portion, and a junction region where the stent-graft portion and the tube-graft portion meet. The stent-graft portion comprises the primary inlet opening and the tube graft portion comprises the primary outlet opening. 22. In some embodiments, the tube-graft portion comprises one or more branch inlet openings configured to fluidly connect the primary lumen and the aortic branches.

In some embodiments, at least one of the one or more branch inlet openings is configured to couple directly to a respective one of the aortic branches.

In some embodiments, the vascular prosthesis further comprises one or more branch conduits in fluid connection with the one or more branch inlet openings. One or more of each of the branch conduits are configured to fluidly connect a respective one of the one or more branch inlet openings to an aortic branch. In some embodiments, each of the branch conduits comprises a branch lumen, a branch outlet opening, and a distal end, each branch lumen configured to extend from a respective branch inlet opening to the respective branch outlet opening, and the distal end of each of the branch conduits is configured to fluidly couple to an aortic branch. In some embodiments, the stent-graft portion comprises a flexible biocompatible membrane attached to a support structure, the stent-graft portion configured to radially expand from a collapsed state to a deployed state. In some embodiments, the vascular prosthesis further comprises a hemostatic apron attached to and circumferentially surrounding the primary conduit, the hemostatic apron configured to be attached to a wall of the aorta of the patient to create an anastomosis between the primary conduit and the aorta to prevent intercostal back bleeding. In some embodiments, the hemostatic apron is a truncated cone-shaped flange that extends from a top end to bottom end, the top end of the hemostatic apron connected to the primary conduit above a junction region and the bottom end of the hemostatic apron located below the junction region. In some embodiments, the vascular prosthesis further comprises a hemostatic collar protruding radially from the primary conduit. In some embodiments, the hemostatic collar comprises interspersed projections or barbs to prevent the vascular prosthesis from migrating within the aorta.

In some embodiments, the system further comprises an inflatable molding balloon configured to be positioned within the primary lumen of the primary conduit.

In some embodiments, the system further comprises one or more package containing the perfusion apparatus, the introducer sheath, and the vascular prosthesis.

In some embodiments, the system further comprises one or more second introducer sheath apparatus. In some embodiments, the system further comprises In some embodiments, the system further comprises. One or more or each of the second introducer sheath apparatuses may be configured to fluidly couple with one of the secondary conduits and deliver blood from the one of the secondary conduits to a respective one of the aortic branches. In some embodiments, the system further comprises introducer sheath apparatus is configured to allow a greater volume of blood flow therethrough at the same pressure than the one or more second introducer sheath apparatuses.

In some embodiments, the system further comprises one or more packages containing the perfusion apparatus, the introducer sheath, vascular prosthesis, and the one or more second introducer sheath.

In some embodiments the disclosure relates to a method of repairing an aortic aneurysm in a subject comprising a heart, an aorta, another inflow artery, and a site of the aortic aneurysm. In some embodiments the method comprises inserting an introducer sheath into the aorta at a site between the heart and the site of the aortic aneurysm or in another inflow artery. In some embodiments, the method further comprises fluidly coupling the introducer sheath to an inlet conduit of a perfusion apparatus. In some embodiments, the inlet conduit comprises a proximal end and the perfusion apparatus further comprises a plurality of secondary conduits branching from the inlet conduit. In some embodiments, one or more or each of the plurality of secondary conduits comprises a respective tip distal to the inlet conduit. The respective tip configured for fluid coupling to aortic branches. In some embodiments, the perfusion apparatus further comprises a valve assembly configured to selectably alter the plurality of secondary conduits between an open flow state and a closed sealed state. In some embodiments, the method further comprises inserting a plurality of second introducer sheath apparatuses into different aortic branches of the subject. In some embodiments, the method further comprises connecting each of the plurality of secondary conduits to a respective one of the plurality of second introducer sheath apparatuses

In some embodiments of the method, the introducer sheath comprises: a hub; a sheath tube extending from the hub, the sheath tube forming a sheath lumen; and a port in fluid cooperation with the sheath lumen. In some embodiments, the step of inserting the introducer sheath comprises inserting the sheath tube, and the step of fluidly coupling the introducer sheath to the inlet conduit comprises coupling the proximal end of the inlet conduit to the port. In some embodiments, one or more or each of the plurality of second introducer sheaths comprise: a second hub; a second sheath tube extending from the second hub, the second sheath tube forming a second sheath lumen; and a second port in fluid cooperation with the sheath lumen. In some embodiments, the step of inserting the plurality of second introducer sheaths comprise inserting each second sheath tube into a respective one of the different aortic branches, In some embodiments, the step of connecting each of the plurality of secondary conduits comprises inserting each tip into a respective second port.

In some embodiments, the disclosure relates to a method of repairing an aortic aneurysm in a subject comprising a heart, an aorta, another inflow artery, and a site of the aortic aneurysm. In some embodiments, the method comprises inserting an introducer sheath into the aorta at a site between the heart and the site of the aortic aneurysm or in the other inflow artery. In some embodiments, the method further comprises fluidly coupling the introducer sheath to an inlet conduit of a perfusion apparatus. In some embodiments, the inlet conduit comprises a proximal end and the perfusion apparatus further comprises a plurality of secondary conduits branching from the inlet conduit. One or more or each secondary conduit comprises a respective tip distal to the inlet conduit configured for fluid coupling to aortic branches. In some embodiments, the a valve assembly is provided and configured to selectably alter the plurality of secondary conduits between an open flow state and a closed sealed state. In some embodiments, the method further comprises connecting each of the plurality of secondary conduits to a respective aortic branch.

In some embodiments, the method further comprises closing the valve assembly to place the plurality of secondary conduits in the closed sealed state prior to the step of fluidly coupling the introducer sheath to the inlet conduit of the perfusion apparatus. In some embodiments, the valve assembly comprises a respective valve for each of the plurality of secondary conduits. In some embodiments, the valve assembly comprises an inlet conduit valve. In some embodiments, the method further comprises selectively opening each of the secondary conduits and the inlet conduit valve after the step of connecting each of the plurality of secondary conduits. In some embodiments, each secondary valve is opened independently only after the step of connecting each of the plurality of secondary conduits is applied to the respective secondary conduit that the secondary valve alters between the open flow state and the closed state.

In some embodiments, the method further comprises opening the inlet conduit valve. In some embodiments, the method further comprises applying a clamp to the aorta between the site of inserting the introducer sheath and the site of the aortic aneurysm. In some embodiments, the method further comprises inserting a vascular prosthesis into the aorta. In some embodiments, the vascular prosthesis spans at least the site of the aortic aneurysm and comprises: a primary conduit forming a primary lumen extending from a primary inlet opening to a primary outlet opening, one or more branch inlet openings, each configured for fluid connection between the primary lumen and the different aortic branches. In some embodiments, the primary conduit is fluidly coupled to the aorta with the primary inlet opening toward the heart.

In some embodiments, the method further comprises directly coupling each of the one or more branch inlet openings to a respective one of the different aortic branches.

In some embodiments of the method, the vascular prosthesis further comprises one or more branch conduits in fluid connection with the one or more branch inlet openings. In some embodiments, one or more or each of the branch conduits comprises a branch lumen, a branch outlet opening, and a distal end opposite to the branch outlet opening. In some embodiments, the method further comprises coupling each distal end to a respective one of the different aortic branches.

In some embodiments, the method further comprises attaching the vascular prosthesis to the aorta.

In some embodiments, the method further comprises removing the introducer sheath and the plurality of second introducer sheaths.

In some embodiments, the method further comprises repairing the site of inserting the vascular prosthesis.

In some embodiments, the method further comprises releasing the clamp.

Some embodiments of the method implements using a system herein.

In some embodiments, the disclosure relates to a system for repairing an aortic aneurysm. In some embodiments, the system comprises a vascular prosthesis. In some embodiments, the vascular prosthesis a primary conduit forming a primary lumen extending from a primary inlet opening to a primary outlet opening, the primary conduit configured to be fluidly coupled to an aorta of a patient. In some embodiments, the system further comprises a perfusion apparatus. In some embodiments, the perfusion apparatus comprises an inlet conduit having a proximal end, and a plurality of secondary conduits branching from the inlet conduit and configured for fluid coupling to aortic branches. In some embodiments, the system further comprises a valve assembly configured to selectably alter the plurality of secondary conduits between an open flow state and a closed sealed state. In some embodiments, the system further comprises an introducer sheath apparatus configured to be fluidly coupled to the inlet conduit of the perfusion apparatus so that blood can flow through the introducer sheath apparatus and into the inlet conduit of the perfusion apparatus.

In some embodiments, the introducer sheath apparatus comprises: a sheath tube for insertion into the aorta or another inflow artery of the patient, the sheath tube forming a working lumen. In some embodiments, the introducer sheath apparatus further comprises a port in fluid cooperation with the working lumen, the port configured to be coupled to the proximal end of the inlet conduit of the perfusion apparatus to fluidly couple the working lumen to the inlet conduit in a sealed manner. In some embodiments, the introducer sheath apparatus further comprises an introducer. In some embodiments, the introducer comprises a shaft portion and a tapered tip portion, and is alterable from: (1) an inserted state in which the tapered tip portion protrudes from a distal end of the sheath tube and the shaft portion is located within the sheath lumen; and (2) a retracted state in which the shaft portion of the introducer is removed from the sheath lumen such that blood can flow through the sheath lumen to the port. In some embodiments, the introducer sheath apparatus further comprises a guide wire for directing the sheath tube within the aorta or the other inflow artery. In some embodiments, the introducer sheath apparatus further comprises a valve that is alterable between: (1) an open state in which blood can flow from the sheath lumen to the port; and (2) a closed state in which blood is prohibited form flowing from the sheath lumen to the port. In some embodiments, the introducer sheath apparatus further comprises a hub, and the sheath tube extends from the hub. In some embodiments, the introducer extends through a passageway in the hub in the inserted state, the introducer comprises a gripping portion that protrudes from the hub of the dilator is in the inserted state. In some embodiments, the port comprises a port connection feature configured to mate with a primary connection feature of the proximal end of the inlet conduit. In some embodiments, the port and primary connection features are selected from a group consisting of bayonet connector component, a threaded connector component, a snap-fit connector component, an interference fit connector component, a luer component, and combinations thereof. In some embodiments, the vascular prosthesis further comprises a plurality of branch conduits, each of the branch conduits forming a branch lumen extending from a branch inlet opening in the primary conduit to a branch outlet opening, the branch inlet opening in fluid communication with the primary lumen, and a distal end of each of the branch conduits configured to be fluidly coupled to an aortic branch. In some embodiments, each of the plurality of secondary conduits of the perfusion apparatus has a distal end configured to be fluidly coupled to one of the aortic branches. In some embodiments, the distal end of each of the secondary conduits of the perfusion apparatus comprises a secondary connection feature configured for fluid coupling to a second introducer sheath apparatus. In some embodiments, the perfusion apparatus comprises four of the secondary conduits. In some embodiments, the valve assembly is configured to be able to individually alter each one of the plurality of secondary conduits between the open flow state and the closed sealed state independent of the other ones of the secondary conduits. In some embodiments, the valve assembly is further configured to be able to alter the inlet conduit of the perfusion apparatus between an open flow state and a closed sealed state. In some embodiments, the valve assembly comprises a plurality of valves, each of the plurality of valves operably coupled to one of the plurality of secondary conduits In some embodiments, each of plurality of valves is a clamp.

In some embodiments, the system further comprises a plurality of the introducer sheath apparatuses. In some embodiments, the port of each of the plurality of the introducer sheath apparatuses comprising a port connection feature. In some embodiments, the proximal end of the inlet port comprises a primary connection feature configured to be fluidly coupled with the port connection feature of one of the plurality of the introducer sheath apparatuses In some embodiments, each of the plurality of secondary conduits of the perfusion apparatus comprise a distal end comprising a second connection feature configured to be connected to one of the plurality of the introducer sheath apparatuses.

In some embodiments of the system, the vascular prosthetic is free of a perfusion conduit.

In some embodiments, the disclosure relates to a medical device for distributing blood from an aorta or another inflow artery of a patient to a plurality aortic branches. In some embodiments, the medical device comprises an introducer sheath sub-system, a perfusion sub-system, and a valve assembly.

In some embodiments, the introducer sheath sub-system comprises a sheath tube forming a working lumen for receiving blood directly from the aorta or the other inflow artery, and a port in fluid cooperation with the working lumen.

In some embodiments, the perfusion sub-system comprises an inlet conduit having a proximal end fluidly coupled to the port of the introducer sheath subs-system to receive blood flowing through the sheath tube from the aorta or the other inflow artery, and a plurality of secondary conduits branching from the inlet conduit. One or more or each of the plurality of secondary conduits comprises a distal end configured for fluid coupling to one of the aortic branches to deliver blood flowing through the inlet conduit to the one of the aortic branches.

In some embodiments, the valve assembly is configured to selectably alter the plurality of secondary conduits between an open flow state and a closed sealed state.

In some embodiments, the introducer sheath sub-system further comprises an introducer comprising a shaft portion and a tapered tip portion, the introducer alterable from: (1) an inserted state in which the tapered tip portion protrudes from a distal end of the sheath tube and the shaft portion is located within the sheath lumen for penetration of a wall of the aorta or the other inflow artery to introduce the sheath tube into the aorta or the other inflow artery; and (2) a retracted state in which the shaft portion of the introducer is removed from the sheath lumen such that blood can flow through the sheath lumen. In some embodiments, the introducer sheath sub-system is separable from the perfusion subsystem. In some embodiments, the introducer sheath subsystem further comprises a valve that is alterable between: (1) an open state in which blood can flow from the sheath lumen to the inlet conduit of the perfusion subsystem; and (2) a closed state in which blood is prohibited form flowing from the sheath lumen to the perfusion subsystem.

In some embodiments, the valve assembly is configured to be able to individually alter each one of the plurality of secondary conduits between the open flow state and the closed sealed state independent of the other ones of the secondary conduits. In some embodiments, the valve assembly is further configured to be able to alter the inlet conduit of the perfusion apparatus between an open flow state and a closed sealed state. In some embodiments, the valve assembly comprises a plurality of valves, each of the plurality of valves operably coupled to one of the plurality of secondary conduits. In some embodiments, each of plurality of valves is a clamp mounted to one of the secondary conduits.

In some embodiments, the disclosure relates to a method of repairing an aortic aneurysm in a damaged section of an aorta. In some embodiments, the method comprises a) preventing the flow of blood in the aorta from reaching the damaged section of the aorta. In some embodiments, the method further comprises b) routing blood from a first section of the aorta that is upstream of the damaged section or from another inflow artery to a plurality of aortic branches via a medical device comprising an introducer sheath sub-system and a perfusion sub-system. In some embodiments, the introducer sheath sub-system receives blood from the first section of the aorta or the other inflow artery via a sheath tube that penetrates an aortic wall of the first section of the aorta or the other inflow artery. In some embodiments, the perfusions subsystem distributes the blood flowing from the sheath tube to a plurality of secondary conduits having distal ends that are fluidly coupled to the plurality of aortic branches In some embodiments, the method comprises c) implanting a vascular prosthetisis in the damaged section.

In some embodiments, the disclosure relates to a medical device for distributing blood from an aorta or another inflow artery of a patient to a plurality aortic branches. In some embodiments, the medical device comprises an introducer sheath sub-system, a perfusion sub-system, and a valve assembly.

In some embodiments, the introducer sheath sub-system comprises a plurality of introducer sheath apparatuses. In some embodiments, one or more or each of the plurality of introducer sheath apparatuses comprises a sheath tube forming a working lumen for flowing blood directly into one of the aortic branches, and a port in fluid cooperation with the working lumen.

In some embodiments, the perfusion sub-system comprises an inlet conduit having a proximal end for receiving blood from the aorta or other inflow artery, and a plurality of secondary conduits branching from the inlet conduit. One or more or each of the plurality of secondary conduits comprise a distal end fluidly coupled to one of the plurality of the introducer sheath apparatuses of the introducer sheath sub-system.

In some embodiments, the valve assembly is configured to selectably alter the plurality of secondary conduits between an open flow state and a closed sealed state.

In some embodiments, the introducer sheath apparatus further comprises an introducer comprising a shaft portion and a tapered tip portion. In some embodiments, the introducer is alterable from: (1) an inserted state in which the tapered tip portion protrudes from a distal end of the sheath tube and the shaft portion is located within the sheath lumen; and (2) a retracted state in which the shaft portion of the introducer is removed from the sheath lumen such that blood can flow through the sheath lumen to the port.

In some embodiments, the disclosure relates to a system for repairing an aortic aneurysm. In some embodiments, the system comprises a vascular prosthesis. In some embodiments, the vascular prosthesis comprises a primary conduit forming a primary lumen extending from a primary inlet opening to a primary outlet opening. The primary conduit is configured to be fluidly coupled to an aorta of a patient. In some embodiments, the perfusion branch conduit forms a perfusion branch lumen extending from a perfusion branch opening in the primary conduit to a perfusion outlet opening at a distal end of the perfusion branch conduit, the perfusion branch opening in fluid communication with the primary lumen.

In some embodiments, the system comprises a perfusion apparatus. In some embodiments, the perfusion apparatus comprises an inlet conduit having a proximal end configured to be fluidly coupled to the distal end of the perfusion branch conduit, and a plurality of secondary conduits branching from the inlet conduit and having a distal end.

In some embodiments, the system comprises a valve assembly configured to selectably alter the plurality of secondary conduits between an open flow state and a closed sealed state.

In some embodiments, the system comprises a plurality of introducer sheath apparatuses. In some embodiments, one or more or each of the plurality of introducer sheath apparatuses are configured to be fluidly coupled to the distal end of one of the plurality of secondary conduits to deliver blood from the secondary conduit to a respective one of the aortic branches.

In some embodiments, one or more or each of the plurality of introducer sheath apparatuses comprises a sheath tube for insertion into one of the aortic branches. In some embodiments, the sheath tube forms a working lumen. In some embodiments, one or more or each of the plurality of introducer sheath apparatuses comprises a port in fluid cooperation with the working lumen, the port configured to be coupled to the distal end of one of the secondary conduits of the perfusion apparatus to fluidly couple the secondary conduit to the working lumen.

In some embodiments, introducer sheath apparatus further comprises an introducer comprising a shaft portion and a tapered tip portion. In some embodiments, the introducer is alterable from: (1) an inserted state in which the tapered tip portion protrudes from a distal end of the sheath tube and the shaft portion is located within the sheath lumen; and (2) a retracted state in which the shaft portion of the introducer is removed from the sheath lumen such that blood can flow through the sheath lumen to the port.

In some embodiments, the distal end of each of the plurality of secondary conduits comprise a second connection feature configured to couple with a port connection feature of the port of one of the plurality of introducer sheath apparatuses.

While embodiments have been described with respect to specific examples, those skilled in the art will appreciate that there are numerous variations and permutations of the above-described embodiments. It is to be understood that other embodiments may be utilized and structural and functional modifications may be made without departing from the scope of the present invention(s).

We claim:

1. A system for repairing an aortic aneurysm in an aorta of a patient, the system comprising:
a perfusion apparatus comprising:
an inlet conduit comprising a proximal end;
a plurality of secondary conduits branching from the inlet conduit and configured for fluid coupling to aortic branches;
a valve assembly configured to selectably alter the plurality of secondary conduits between an open flow state and a closed sealed state; and
an introducer sheath apparatus configured to be fluidly coupled to the inlet conduit of the perfusion apparatus to deliver blood from the aorta or another inflow artery to the inlet conduit.

2. The system of claim 1, wherein the introducer sheath apparatus comprises:
a sheath tube configured for insertion into the aorta or the other inflow artery, the sheath tube forming a working lumen; and
a port in fluid cooperation with the sheath lumen, the port configured to be fluidly coupled to the proximal end of the inlet conduit of the perfusion apparatus to fluidly couple the working lumen to the inlet conduit.

3. The system according to any one of claims 1 to 2, wherein the introducer sheath apparatus further comprises a valve configured to selectively prevent flow through the introducer sheath apparatus to the inlet conduit.

4. The system according to any one of claims 1 to 3, wherein the introducer sheath apparatus further comprises one or more of a dilator and a guide wire.

5. The system according to any one of claims 2 to 5, wherein the port of the introducer sheath apparatus comprises a port connection feature for coupling to a primary connection feature of the proximal end of the inlet conduit.

6. The system according to any one of claims 1 to 6, wherein each of the plurality of secondary conduits of the perfusion apparatus has a distal end configured to be fluidly coupled to one of the aortic branches.

7. The system according to claim 6, further comprising a balloon fixed to the distal ends of each of the plurality of secondary conduits, the balloon configured to tight fit in the respective aortic branch in which it is configured to be inserted.

8. The system according to any one of claims 2 to 4 further comprising:
a plurality of the introducer sheath apparatuses, the port of each of the plurality of the introducer sheath apparatuses comprising a port connection feature;
the proximal end of the inlet port comprising a primary connection feature configured to be fluidly coupled with the port connection feature of one of the plurality of the introducer sheath apparatuses; and
each of the plurality of secondary conduits of the perfusion apparatus having a distal end comprising a second connection feature configured to be connected to one of the plurality of the introducer sheath apparatuses.

9. The system according to claim 8, wherein the primary connection feature and the second connection features are the same such that the primary connection feature can be fluidly coupled to any one of the port connection features of the plurality of the introducer sheath apparatuses and the second connection features can be fluidly coupled to any one of the port connection features of the plurality of the introducer sheath apparatuses.

10. The system according to claim 8, wherein the primary connection feature is different from the second connection features such that the primary connection feature and the secondary connection features cannot be fluidly coupled to the same port connection features of the plurality of the introducer sheath apparatuses.

11. The system according to any one of claims 8 and 10, wherein the port connection feature of at least one of the plurality of the introducer sheath apparatuses is configured to be fluidly couplable to the primary connection feature but not fluidly couplable to the secondary connection features; and wherein the port connection feature of the remaining plurality of the introducer sheath apparatuses are configured to be fluidly couplable to the secondary connection features but not fluidly couplable to the primary connection feature.

12. The system according to claim 11, wherein the primary connection feature is at least one of: (i) a different size than the secondary connection features; (ii) a different type than the secondary connection feature; and (iii) has a different structure than the secondary connection features.

13. The system according to any one of claims 8 to 11 wherein at least one of the plurality of the introducer sheath apparatuses is configured to allow a greater volume of blood flow therethrough at the same pressure than other ones of the plurality of the introducer sheath apparatuses.

14. The system according to any one of claims 1 to 13 wherein the wherein the perfusion apparatus comprises four of the secondary conduits.

15. The system according to any one of claims 1 to 14 wherein the valve assembly is configured to individually alter each one of the plurality of secondary conduits between the open flow state and the closed sealed state independent of the other ones of the secondary conduits.

16. The system according to any one of claims 1 to 15 wherein the valve assembly comprises a plurality of valves, each of the plurality of valves operably coupled to one of the plurality of secondary conduits.

17. The system according to claim 16 wherein each of plurality of valves is a clamp.

18. The system according to any one of claims 1 to 17 further comprising one or more a sealed packages containing the perfusion apparatus and the introducer sheath.

19. The system according to any one of claims 1 to 18, wherein the introducer sheath apparatus further comprises an introducer comprising a shaft portion and a tapered tip portion, the introducer alterable from: (1) an inserted state in which the tapered tip portion protrudes from a distal end of a sheath tube of the introducer sheath apparatus and the shaft portion is located within a sheath lumen of the sheath tube for penetration of a wall of the aorta or the other inflow artery to introduce the sheath tube into the aorta or the other inflow artery; and (2) a retracted state in which the shaft portion of the introducer is removed from the sheath lumen such that blood can flow through the sheath lumen.

20. The system according to any one of claims 1 to 19 further comprising a vascular prosthesis comprising a primary conduit forming a primary lumen extending from a primary inlet opening to a primary outlet opening, the primary conduit configured to be fluidly coupled to the aorta of the patient.

21. The system according to claim 20, wherein the vascular prosthesis further comprises a stent-graft portion, a tube-graft portion, and a junction region where the stent-graft portion and the tube-graft portion meet, the stent-graft portion comprising the primary inlet opening and the tube graft portion comprising the primary outlet opening.

22. The system according to claim 21, wherein the tube-graft portion comprises one or more branch inlet openings configured to fluidly connect the primary lumen and the aortic branches.

23. The system according to claim 22, wherein at least one of the one or more branch inlet openings is configured to couple directly to a respective one of the aortic branches.

24. The system according to any one of claims 20 to 21, wherein the vascular prosthesis further comprises one or more branch conduits in fluid connection with the one or more branch inlet openings and each configured to fluidly connect a respective one of the one or more branch inlet openings to an aortic branch.

25. The system of claim 16, wherein each of the branch conduits comprises a branch lumen, a branch outlet opening, and a distal end, each branch lumen configured to extend from a respective branch inlet opening to the respective branch outlet opening, and the distal end of each of the branch conduits is configured to fluidly couple to an aortic branch.

26. The system of any of claims 21 to 25, wherein the stent-graft portion comprises a flexible biocompatible membrane attached to a support structure, the stent-graft portion configured to radially expand from a collapsed state to a deployed state.

27. The system of any of claims 20 to 26, wherein the vascular prosthesis further comprises a hemostatic apron attached to and circumferentially surrounding the primary conduit, the hemostatic apron configured to be attached to a wall of the aorta of the patient to create an anastomosis between the primary conduit and the aorta to prevent intercostal back bleeding.

28. The system of claim 27 wherein the hemostatic apron is a truncated cone-shaped flange that extends from a top end to bottom end, the top end of the hemostatic apron connected to the primary conduit above a junction region and the bottom end of the hemostatic apron located below the junction region.

29. The system of any one of claims 20 to 28 wherein the vascular prosthesis further comprises a hemostatic collar protruding radially from the primary conduit.

30. The system of claim 29 wherein the hemostatic collar comprises interspersed projections or barbs to prevent the vascular prosthesis from migrating within the aorta.

31. The system of any one of claims 20 to 30 further comprising an inflatable molding balloon configured to be positioned within the primary lumen of the primary conduit.

32. The system of any of claims 20 to 31 further comprising one or more package containing the perfusion apparatus, the introducer sheath, and the vascular prosthesis.

33. The system of claim 1 further comprising one or more second introducer sheath apparatus, each second introducer sheath apparatus configured to fluidly couple with one of the secondary conduits and deliver blood from the one of the secondary conduits to a respective one of the aortic branches.

34. The system of claim 33 wherein the introducer sheath apparatus is configured to allow a greater volume of blood flow therethrough at the same pressure than the one or more second introducer sheath apparatuses.

35. The system of claim 33 further comprising one or more packages containing the perfusion apparatus, the introducer sheath, vascular prosthesis, and the one or more second introducer sheath.

36. A method of repairing an aortic aneurysm in a subject comprising a heart, an aorta, another inflow artery, and a site of the aortic aneurysm, the method comprising:
inserting an introducer sheath into the aorta at a site between the heart and the site of the aortic aneurysm or into the other inflow artery,
fluidly coupling the introducer sheath to an inlet conduit of a perfusion apparatus; the inlet conduit comprising a proximal end and the perfusion apparatus further comprising a plurality of secondary conduits branching from the inlet conduit, each comprising a respective tip distal to the inlet conduit, and configured for fluid coupling to aortic branches, and a valve assembly configured to selectably alter the plurality of secondary conduits between an open flow state and a closed sealed state,
inserting a plurality of second introducer sheath apparatuses into different aortic branches of the subject,
connecting each of the plurality of secondary conduits to a respective one of the plurality of second introducer sheath apparatuses.

37. The method of claim 36, wherein the introducer sheath comprises:
a hub;
a sheath tube extending from the hub, the sheath tube forming a sheath lumen; and
a port in fluid cooperation with the sheath lumen,
wherein the step of inserting the introducer sheath comprises inserting the sheath tube, and
the step of fluidly coupling the introducer sheath to the inlet conduit comprises coupling the proximal end of the inlet conduit to the port.

38. The method of claim 36 or 37, wherein each of the plurality of second introducer sheaths comprise:
a second hub;
a second sheath tube extending from the second hub, the second sheath tube forming a second sheath lumen; and
a second port in fluid cooperation with the sheath lumen,
wherein the step of inserting the plurality of second introducer sheaths comprise inserting each second sheath tube into a respective one of the different aortic branches, and
the step of connecting each of the plurality of secondary conduits comprises inserting each tip into a respective second port.

39. A method of repairing an aortic aneurysm in a subject comprising a heart, an aorta, another inflow artery, and a site of the aortic aneurysm, the method comprising:
inserting an introducer sheath into the aorta at a site between the heart and the site of the aortic aneurysm or into the other inflow artery,
fluidly coupling the introducer sheath to an inlet conduit of a perfusion apparatus; the inlet conduit comprising a proximal end and the perfusion apparatus further comprising a plurality of secondary conduits branching from the inlet conduit, each comprising a respective tip distal to the inlet conduit, and configured for fluid coupling to aortic branches, and a valve assembly configured to selectably alter the plurality of secondary conduits between an open flow state and a closed sealed state,
connecting each of the plurality of secondary conduits to a respective aortic branch.

40. The method of any of claims 36 to 39 further comprising closing the valve assembly to place the plurality of secondary conduits in the closed sealed state prior to the step of fluidly coupling the introducer sheath to the inlet conduit of the perfusion apparatus.

41. The method of any of claims 36 to 40, wherein the valve assembly comprises a respective valve for each of the plurality of secondary conduits.

42. The method of any of claims 36 to 41, wherein the valve assembly comprises an inlet conduit valve.

43. The method of claim 41 or 42 further comprising selectively opening each of the secondary conduits and the inlet conduit valve after the step of connecting each of the plurality of secondary conduits.

44. The method of claim 43, wherein each secondary valve is opened independently only after the step of connecting each of the plurality of secondary conduits is applied to the respective secondary conduit that the secondary valve alters between the open flow state and the closed state.

45. The method of any of claims 41 to 43 further comprising opening the inlet conduit valve.

46. The method of any one of claims 36 to 45 further comprising applying a clamp to the aorta between the site of inserting the introducer sheath and the site of the aortic aneurysm.

47. The method of claim 46 further comprising inserting a vascular prosthesis into the aorta, wherein the vascular prosthesis spans at least the site of the aortic aneurysm and comprises: a primary conduit forming a primary lumen extending from a primary inlet opening to a primary outlet opening, one or more branch inlet openings, each configured for fluid connection between the primary lumen and the different aortic branches, wherein the primary conduit is fluidly coupled to the aorta opening toward the heart.

48. The method of claim 47 further comprising directly coupling each of the one or more branch inlet openings to a respective one of the different aortic branches.

49. The method of claim 47, wherein the vascular prosthesis further comprises one or more branch conduits in fluid connection with the one or more branch inlet openings, each of the branch conduits comprising a branch lumen, a branch outlet opening, and a distal end opposite to the branch outlet opening, the method further comprising coupling each distal end to a respective one of the different aortic branches.

50. The method of any of claims 47 to 49 further comprising attaching the vascular prosthesis to the aorta.

51. The method of any one of claims 48 to 44 further comprising removing the introducer sheath and the plurality of second introducer sheaths.

52. The method of any of claims 48 to 51 further comprising repairing the site of inserting the vascular prosthesis.

53. The method of claim 51 or 52 further comprising releasing the clamp.

54. The method of any one of claims 36 to 53 comprising implementing the system of any of claims 1-30.

55. A system for repairing an aortic aneurysm comprising:
a vascular prosthesis comprising:
a primary conduit forming a primary lumen extending from a primary inlet opening to a primary outlet opening, the primary conduit configured to be fluidly coupled to an aorta of a patient;
a perfusion apparatus comprising:
an inlet conduit having a proximal end;
a plurality of secondary conduits branching from the inlet conduit and configured for fluid coupling to aortic branches;
a valve assembly configured to selectably alter the plurality of secondary conduits between an open flow state and a closed sealed state; and
an introducer sheath apparatus configured to be fluidly coupled to the inlet conduit of the perfusion apparatus so that blood can flow through the introducer sheath apparatus and into the inlet conduit of the perfusion apparatus.

56. The system of claim 55 wherein the introducer sheath apparatus comprises:
a sheath tube for insertion into the aorta or another inflow artery of the patient, the sheath tube forming a working lumen; and
a port in fluid cooperation with the working lumen, the port configured to be coupled to the proximal end of the inlet conduit of the perfusion apparatus to fluidly couple the working lumen to the inlet conduit in a sealed manner.

57. The system of claim 56 wherein the introducer sheath apparatus further comprises an introducer comprising a shaft portion and a tapered tip portion, the introducer alterable from: (1) an inserted state in which the tapered tip portion protrudes from a distal end of the sheath tube and the shaft portion is located within the sheath lumen; and (2) a retracted state in which the shaft portion of the introducer is removed from the sheath lumen such that blood can flow through the sheath lumen to the port.

58. The system of claim 56 or 57 wherein the introducer sheath apparatus further comprises a guide wire for directing the sheath tube within the aorta or the other inflow artery.

59. The system of any one of claims 56 to 58 wherein the introducer sheath apparatus further comprises a valve that is alterable between: (1) an open state in which blood can flow from the sheath lumen to the port; and (2) a closed state in which blood is prohibited form flowing from the sheath lumen to the port.

60. The system of any one of claims 56 to 59 wherein the introducer sheath apparatus further comprises a hub, the sheath tube extending from the hub; and wherein the introducer extends through a passageway in the hub in the inserted state, the introducer comprising a gripping portion that protrudes from the hub of the dilator is in the inserted state.

61. The system of any one of claims 56 to 60 wherein the port comprises a port connection feature configured to mate with a primary connection feature of the proximal end of the inlet conduit.

62. The system of claim 61 wherein the port and primary connection features are selected from a group consisting of bayonet connector component, a threaded connector component, a snap-fit connector component, an interference fit connector component, a luer component, and combinations thereof.

63. The system of any one of claims 56 to 62 wherein the vascular prosthesis further comprises a plurality of branch conduits, each of the branch conduits forming a branch lumen extending from a branch inlet opening in the primary conduit to a branch outlet opening, the branch inlet opening in fluid communication with the primary lumen, and a distal end of each of the branch conduits configured to be fluidly coupled to an aortic branch.

64. The system of any one of claims 55 to 63 wherein each of the plurality of secondary conduits of the perfusion apparatus has a distal end configured to be fluidly coupled to one of the aortic branches.

65. The system of claim 64 wherein the distal end of each of the secondary conduits of the perfusion apparatus comprises a secondary connection feature configured for fluid coupling to a second introducer sheath apparatus.

66. The system of any one of claims 55 to 65 wherein the perfusion apparatus comprises four of the secondary conduits.

67. The system of any one of claims 55 to 66 wherein the valve assembly is configured to be able to individually alter each one of the plurality of secondary conduits between the open flow state and the closed sealed state independent of the other ones of the secondary conduits.

68. The system of claim 67 wherein the valve assembly is further configured to be able to alter the inlet conduit of the perfusion apparatus between an open flow state and a closed sealed state.

69. The system of claim 67 or 68 wherein the valve assembly comprises a plurality of valves, each of the plurality of valves operably coupled to one of the plurality of secondary conduits.

70. The system of claim 69 wherein each of plurality of valves is a clamp.

71. The system of any one of claims 55 to 70 further comprising:
a plurality of the introducer sheath apparatuses, the port of each of the plurality of the introducer sheath apparatuses comprising a port connection feature;
the proximal end of the inlet port comprising a primary connection feature configured to be fluidly coupled with the port connection feature of one of the plurality of the introducer sheath apparatuses; and
each of the plurality of secondary conduits of the perfusion apparatus having a distal end comprising a second connection feature configured to be connected to one of the plurality of the introducer sheath apparatuses.

72. The system of any one of claims 55 to 71 wherein the vascular prosthetic is free of a perfusion conduit.

73. A medical device for distributing blood from an aorta or another inflow artery of a patient to a plurality aortic branches comprising:
an introducer sheath sub-system comprising:
a sheath tube forming a working lumen for receiving blood directly from the aorta or the other inflow artery;
a port in fluid cooperation with the working lumen;
a perfusion sub-system comprising:
an inlet conduit having a proximal end fluidly coupled to the port of the introducer sheath subs-system to receive blood flowing through the sheath tube from the aorta or the other inflow artery;
a plurality of secondary conduits branching from the inlet conduit, each of the plurality of secondary conduits comprising a distal end configured for fluid coupling to one of the aortic branches to deliver blood flowing through the inlet conduit to the one of the aortic branches;
a valve assembly configured to selectably alter the plurality of secondary conduits between an open flow state and a closed sealed state.

74. The medical device of claim 73 wherein the introducer sheath sub-system further comprises an introducer comprising a shaft portion and a tapered tip portion, the introducer alterable from: (1) an inserted state in which the tapered tip portion protrudes from a distal end of the sheath tube and the shaft portion is located within the sheath lumen for penetration of a wall of the aorta or the other inflow artery to introduce the sheath tube into the aorta or the other inflow artery; and (2) a retracted state in which the shaft portion of the introducer is removed from the sheath lumen such that blood can flow through the sheath lumen.

75. The medical device of claim 73 to 74 wherein the introducer sheath sub-system is separable from the perfusion subsystem.

76. The medical device of any one of claims 73 to 75 wherein the introducer sheath subsystem further comprises a valve that is alterable between: (1) an open state in which blood can flow from the sheath lumen to the inlet conduit of the perfusion subsystem; and (2) a closed state in which blood is prohibited form flowing from the sheath lumen to the perfusion subsystem.

77. The medical device of any one of claims 73 to 76 wherein the valve assembly is configured to be able to individually alter each one of the plurality of secondary conduits between the open flow state and the closed sealed state independent of the other ones of the secondary conduits.

78. The medical device of claim 77 wherein the valve assembly is further configured to be able to alter the inlet conduit of the perfusion apparatus between an open flow state and a closed sealed state.

79. The medical device of claims 77 or 78 wherein the valve assembly comprises a plurality of valves, each of the plurality of valves operably coupled to one of the plurality of secondary conduits.

80. The medical device of claim 79 wherein each of plurality of valves is a clamp mounted to one of the secondary conduits.

81. A method of repairing an aortic aneurysm in a damaged section of an aorta, the method comprising:
a) preventing the flow of blood in the aorta from reaching the damaged section of the aorta;
b) routing blood from a first section of the aorta or another inflow artery that is upstream of the damaged section to a plurality of aortic branches via a medical device comprising an introducer sheath sub-system and a perfusion sub-system, the introducer sheath sub-system receiving blood from the first section of the aorta or the other inflow artery via a sheath tube that penetrates an aortic wall of the first section of the aorta or the other inflow artery wall and the perfusions subsystem distributing the blood flowing from the sheath tube to a plurality of secondary conduits having distal ends that are fluidly coupled to the plurality of aortic branches; and
c) implanting a vascular prosthetisis in the damaged section.

82. A medical device for distributing blood from an aorta or another inflow artery of a patient to a plurality aortic branches comprising:
an introducer sheath sub-system comprising a plurality of introducer sheath apparatuses, each of the plurality of introducer sheath apparatuses comprising:
a sheath tube forming a working lumen for flowing blood directly into one of the aortic branches;
a port in fluid cooperation with the working lumen;
a perfusion sub-system comprising:
an inlet conduit having a proximal end for receiving blood from the aorta or the other inflow artery; and
a plurality of secondary conduits branching from the inlet conduit, each of the plurality of secondary conduits comprising a distal end fluidly coupled to one of the plurality of the introducer sheath apparatuses of the introducer sheath sub-system; and
a valve assembly configured to selectably alter the plurality of secondary conduits between an open flow state and a closed sealed state.

83. The medical device according to claim 82 wherein the introducer sheath apparatus further comprises an introducer comprising a shaft portion and a tapered tip portion, the introducer alterable from: (1) an inserted state in which the tapered tip portion protrudes from a distal end of the sheath tube and the shaft portion is located within the sheath lumen; and (2) a retracted state in which the shaft portion of the introducer is removed from the sheath lumen such that blood can flow through the sheath lumen to the port.

84. A system for repairing an aortic aneurysm comprising:
a vascular prosthesis comprising:
a primary conduit forming a primary lumen extending from a primary inlet opening to a primary outlet opening, the primary conduit configured to be fluidly coupled to an aorta of a patient; and
a perfusion branch conduit forming a perfusion branch lumen extending from a perfusion branch opening in the primary conduit to a perfusion outlet opening at a distal end of the perfusion branch conduit, the perfusion branch opening in fluid communication with the primary lumen;
a perfusion apparatus comprising:
an inlet conduit having a proximal end configured to be fluidly coupled to the distal end of the perfusion branch conduit;
a plurality of secondary conduits branching from the inlet conduit and having a distal end;
a valve assembly configured to selectably alter the plurality of secondary conduits between an open flow state and a closed sealed state; and
a plurality of introducer sheath apparatuses, each of the plurality of introducer sheath apparatuses configured to be fluidly coupled to the distal end of one of the plurality of secondary conduits to deliver blood from the secondary conduit to a respective one of the aortic branches.

85. The system of claim 84, wherein each of the plurality of introducer sheath apparatuses comprises:
a sheath tube for insertion into one of the aortic branches, the sheath tube forming a working lumen; and
a port in fluid cooperation with the working lumen, the port configured to be coupled to the distal end of one of the secondary conduits of the perfusion apparatus to fluidly couple the secondary conduit to the working lumen.

86. The system of claim 85 wherein the introducer sheath apparatus further comprises an introducer comprising a shaft portion and a tapered tip portion, the introducer alterable from: (1) an inserted state in which the tapered tip portion protrudes from a distal end of the sheath tube and the shaft portion is located within the sheath lumen; and (2) a retracted state in which the shaft portion of the introducer is removed from the sheath lumen such that blood can flow through the sheath lumen to the port.

87. The system of claim 85 or 86, wherein the distal end of each of the plurality of secondary conduits comprise a second connection feature configured to couple with a port connection feature of the port of one of the plurality of introducer sheath apparatuses.
